(19) United States
(12) Patent Application Publication
(10) Pub. No.: US 2026/0130657 A1
(43) Pub. Date: May 14, 2026

(54) TRANSMISSION FOR A MOTOR-POWERED DEVICE WITH SELECTABLE OUTPUTS

(71) Applicant: CILAG GMBH INTERNATIONAL, ZUG (CH)

(72) Inventors: FREDERICK E. SHELTON, HILLSBORO, OH (US); RICHARD TIMM, CINCINNATI, OH (US); MATTHEW D. COWPERTHWAIT, CINCINNATI, OH (US); KARL W. MUELLER, CINCINNATI, OH (US); SHANE R. ADAMS, LEBANON, OH (US); THOMAS ALLEN STOUGHTON, CINCINNATI, OH (US); SETH HOLDMEYER, CINCINNATI, OH (US); ADAM HENSEL, CINCINNATI, OH (US); ANDREW DECK, CINCINNATI, OH (US)

(73) Assignee: CILAG GMBH INTERNATIONAL, ZUG (CH)

(21) Appl. No.: 19/380,578

(22) Filed: Nov. 5, 2025

Related U.S. Application Data
(60) Provisional application No. 63/717,951, filed on Nov. 8, 2024.

(51) Int. Cl. A61B 17/00; F16H 1/46; F16H 57/10

(52) U.S. Cl. CPC A61B 17/00; F16H 1/46; F16H 57/10; A61B 2017/00398

(57) ABSTRACT

A transmission for a motor-powered device is herein disclosed. The transmission includes a motor input, a first output and a second output both drivable by the motor input, and a planetary gearbox. The planetary gearbox includes a first stage ring gear engaged with the first output, a second stage ring gear engaged with the second output, and a lock selectively engageable with at least one of the first stage ring gear or the second stage ring gear, the lock having an unlocked position allowing the at least one of the first stage ring gear or the second stage ring gear to power at least one of the first output or the second output and a locked position that stops rotation of the at least one of the first stage ring gear or the second stage ring gear to lock at least one of the first output or the second output.

### CROSS-REFERENCE TO RELATED APPLICATIONS

This application claims the benefit of and priority to U.S. Provisional Patent Application No. 63/717,951 filed Nov. 8, 2024 (Attorney Docket END9648USPSP1_264142.172), the disclosure of which is expressly incorporated herein by reference.

### FIELD OF THE INVENTION

The present disclosure relates to motor-powered devices including a transmission and, in various arrangements, to surgical stapling and cutting instruments including a transmission that are designed to staple and cut tissue.

### BACKGROUND

Surgical staplers are often used to deploy staples into soft tissue to reduce or eliminate bleeding from the soft tissue, especially as the tissue is being transected, for example. Surgical staplers, such as an endocutter, for example, can comprise an end effector which can be moved, or articulated, with respect to an elongated shaft assembly. End effectors are often configured to secure soft tissue between first and second jaw members where the first jaw member often includes a staple cartridge which is configured to removably store staples therein and the second jaw member often includes an anvil. Such surgical staplers can include a closing system for pivoting the anvil relative to the staple cartridge.

Surgical staplers, as outlined above, can be configured to pivot the anvil of the end effector relative to the staple cartridge in order to capture soft tissue therebetween. In various circumstances, the anvil can be configured to apply a clamping force to the soft tissue in order to hold the soft tissue tightly between the anvil and the staple cartridge. If a surgeon is unsatisfied with the position of the end effector, however, the surgeon must typically activate a release mechanism on the surgical stapler to pivot the anvil into an open position and then reposition the end effector. Thereafter, staples are typically deployed from the staple cartridge by a driver which traverses a channel in the staple cartridge and causes the staples to be deformed against the anvil and secure layers of the soft tissue together. Often, as known in the art, the staples are deployed in several staple lines, or rows, in order to more reliably secure the layers of tissue together. The end effector may also include a cutting member, such as a knife, for example, which is advanced between two rows of the staples to resect the soft tissue after the layers of the soft tissue have been stapled together.

These surgical staplers, and other motor-powered devices, typically employ either employ multiple motors and/or complex, costly shifting systems to power the different outputs. It would be beneficial, therefore, to provide a more simplified system to permit switching between different outputs in a motor-powered device.

The foregoing discussion is intended only to illustrate various aspects of the related art in the field of this disclosure at the time, and should not be taken as a disavowal of claim scope.

### SUMMARY

The disclosed technology can be for systems, devices, and subsystems for motor-powered devices.

The disclosed technology describes a transmission, which can be one of a number of subsystems and/or subcomponents for a motor-powered device. The transmission includes a motor input, a first output drivable by the motor input, a second output drivable by the motor input, and a planetary gearbox operably connecting the motor input with the first output and the second output. The planetary gearbox includes a first stage ring gear engaged with the first output, a second stage ring gear engaged with the second output, and a lock selectively engageable with at least one of the first stage ring gear or the second stage ring gear. The lock has (i) an unlocked position allowing the at least one of the first stage ring gear or the second stage ring gear to power at least one of the first output or the second output and (ii) a locked position that stops rotation of the at least one of the first stage ring gear or the second stage ring gear to lock at least one of the first output or the second output.

The disclosed technology describes a surgical instrument comprising the transmission described in the foregoing paragraph.

The disclosed technology describes a method of operating a motor-powered device. The method includes controlling a lock such that the lock is engaged with a first stage ring gear and disengaged with a second stage ring gear. The method includes powering a first output engaged with the first stage ring gear. The method includes locking a second output engaged with the second stage ring gear. The method includes controlling (1008) the lock such that the lock is engaged with the second stage ring gear and disengaged with the first stage ring gear. The method includes powering the second output. The method includes locking the first output.

### BRIEF DESCRIPTION OF THE DRAWINGS

Various features of the examples described herein, together with advantages thereof, may be understood in accordance with the following description taken in conjunction with the accompanying drawings as follows:

FIG. 1 is a schematic pictorial illustration of a block diagram of an exemplary motor-powered device with a transmission, in accordance with the presently disclosed technology;

FIG. 2A is a schematic pictorial illustration of a block diagram of the exemplary motor-powered device with the transmission of FIG. 1, showing an exemplary use thereof, in accordance with the presently disclosed technology;

FIG. 2B is a schematic pictorial illustration of a block diagram of the exemplary motor-powered device with the transmission of FIG. 1, showing another exemplary use thereof, in accordance with the presently disclosed technology;

FIG. 2C is a schematic pictorial illustration of a block diagram of the exemplary motor-powered device with the transmission of FIG. 1, showing another exemplary use thereof, in accordance with the presently disclosed technology;

FIG. 3 is a perspective view of an exemplary handheld powered surgical instrument, in accordance with the presently disclosed technology;

FIG. 4 is a schematic pictorial illustration of a partial cross-sectional view of the surgical instrument of FIG. 3 taken along a longitudinal axis thereof, in accordance with the presently disclosed technology;

FIG. 5A is a schematic pictorial illustration of a detail view of a motor, transmission, and lock of the surgical instrument of FIG. 3, in accordance with the presently disclosed technology;

FIG. 5B is a schematic pictorial illustration of another detail view of the motor, transmission, and lock of FIG. 5A, in accordance with the presently disclosed technology;

FIG. 6A is a schematic pictorial illustration of a detail elevation view of a stage and lock of the transmission of FIG. 4, in accordance with the presently disclosed technology;

FIG. 6B is a schematic pictorial illustration of a detail elevation view of a modified stage and lock of a transmission, in accordance with the presently disclosed technology;

FIG. 6C is a schematic pictorial illustration of a detail elevation view of another modified stage and lock of a transmission, in accordance with the presently disclosed technology;

FIG. 6D is a schematic pictorial illustration of a detail elevation view of another modified stage and lock of a transmission, in accordance with the presently disclosed technology;

FIG. 6E is a schematic pictorial illustration of a detail elevation view of another modified stage and lock of a transmission, in accordance with the presently disclosed technology;

FIG. 6F is a schematic pictorial illustration of a detail elevation view of another modified stage and lock of a transmission, in accordance with the presently disclosed technology;

FIG. 7A is schematic pictorial illustration of a detail elevation view of another motor, transmission, and lock in a first position, in accordance with the presently disclosed technology;

FIG. 7B is a schematic pictorial illustration of a partial cross-sectional view of the motor, transmission, and lock of FIG. 7A taken along a longitudinal axis thereof, in accordance with the presently disclosed technology;

FIG. 7C is schematic pictorial illustration of a detail elevation view of the another motor, transmission, and lock in a second position, in accordance with the presently disclosed technology;

FIG. 7D is a schematic pictorial illustration of a partial cross-sectional view of the motor, transmission, and lock of FIG. 7C taken along the longitudinal axis thereof, in accordance with the presently disclosed technology;

FIG. 8A is a schematic pictorial illustration of a detail cross-sectional view of a portion of the lock and transmission of FIG. 7B, showing engagement of the lock and transmission;

FIG. 8B is a schematic pictorial illustration of a detail cross-sectional view of a portion of the lock and transmission of FIG. 7D, showing engagement of the lock and transmission;

FIG. 9 is a flow diagram of a method of operating a motor-powered device, in accordance with the disclosed technology;

FIG. 10 is a schematic pictorial illustration of a partial elevation view of yet another modified lock and transmission in a first position, in accordance with the presently disclosed technology;

FIG. 11 is a schematic pictorial illustration of a partial cross-sectional view of the modified lock and transmission of FIG. 10 taken along line 11-11 in FIG. 10, in accordance with the presently disclosed technology;

FIG. 12 is a schematic pictorial illustration of a partial elevation view of the modified lock and transmission of FIG. 10 in a second position, in accordance with the presently disclosed technology;

FIG. 13 is a schematic pictorial illustration of a partial cross-sectional view of the modified lock and transmission of FIG. 12 taken along line 13-13 in FIG. 12, in accordance with the presently disclosed technology;

FIG. 14 is a schematic pictorial illustration of a partial elevation view of the modified lock and transmission of FIG. 10 in a third position, in accordance with the presently disclosed technology;

FIG. 15 is a schematic pictorial illustration of a partial cross-sectional view of the modified lock and transmission of FIG. 14 taken along line 15-15 in FIG. 14, in accordance with the presently disclosed technology;

FIG. 16 is a schematic pictorial illustration of a partial cross-sectional view of yet another modified lock and transmission, in accordance with the presently disclosed technology; and

FIG. 17 is a schematic pictorial illustration of a partial cross-sectional view of the modified lock and transmission of FIG. 16 taken along line 17-17 in FIG. 16, in accordance with the presently disclosed technology.

Corresponding reference characters indicate corresponding parts throughout the several views. The exemplifications set out herein illustrate various examples of the disclosed technology, in one form, and such exemplifications are not to be construed as limiting the scope of the disclosed technology in any manner.

### DETAILED DESCRIPTION

Numerous specific details are set forth to provide a thorough understanding of the overall structure, function, manufacture, and use of the embodiments as described in the specification and illustrated in the accompanying drawings. Well-known operations, components, and elements have not been described in detail so as not to obscure the embodiments described in the specification. The reader will understand that the embodiments described and illustrated herein are non-limiting examples, and thus it can be appreciated that the specific structural and functional details disclosed herein may be representative and illustrative. Variations and changes thereto may be made without departing from the scope of the claims.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a system, device, or apparatus that “comprises,” “has,” “includes” or “contains” one or more elements possesses those one or more elements, but is not limited to possessing only those one or more elements. Likewise, an element of a system, device, or apparatus that “comprises,” “has,” “includes” or “contains” one or more features possesses those one or more features, but is not limited to possessing only those one or more features.

The terms “proximal” and “distal” are used herein with reference to a user (e.g., a clinician) manipulating the handle portion of a motor-powered device (e.g., a surgical instrument). The term “proximal” refers to the portion closest to the user and the term “distal” refers to the portion located away from the user. It will be further appreciated that, for convenience and clarity, spatial terms such as “vertical”, “horizontal”, “up”, and “down” may be used herein with respect to the drawings. However, motor-powered devices are used in many orientations and positions, and these terms are not intended to be limiting and/or absolute.

As those skilled in the art will appreciate, the motor-powered device that is described in the following paragraphs can be embodied in any number of appropriate forms. While primarily described in the context of a surgical instrument (e.g., a handheld or robotically controlled surgical stapler/endocutter), the transmission of the present disclosure can be implemented in various other devices, such as, but not limited to, power tools, industrial machinery, robots, and other precision/medical devices not explicitly mentioned, without departing from the spirit and scope of the present disclosure.

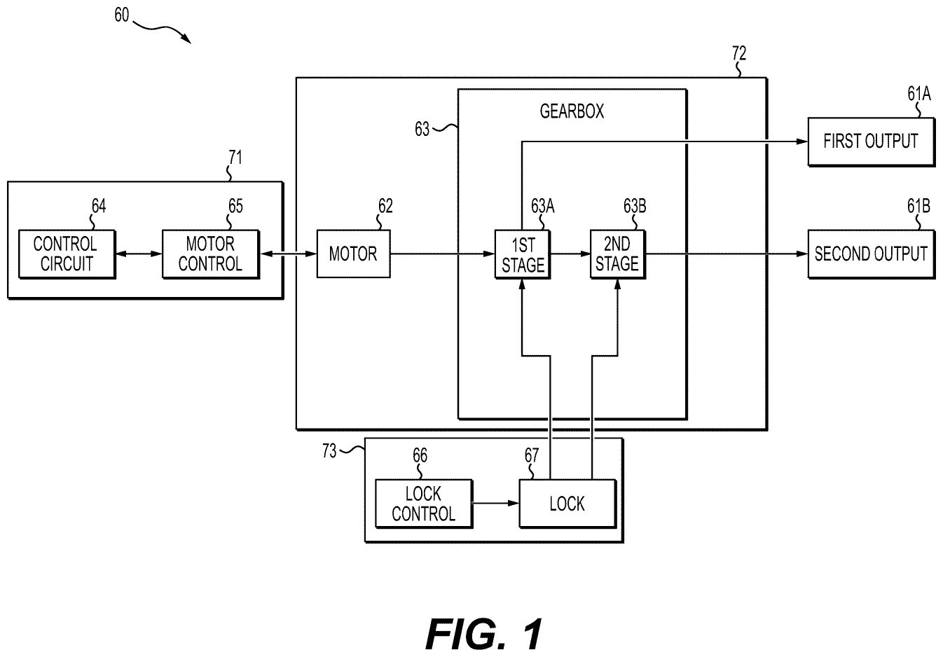

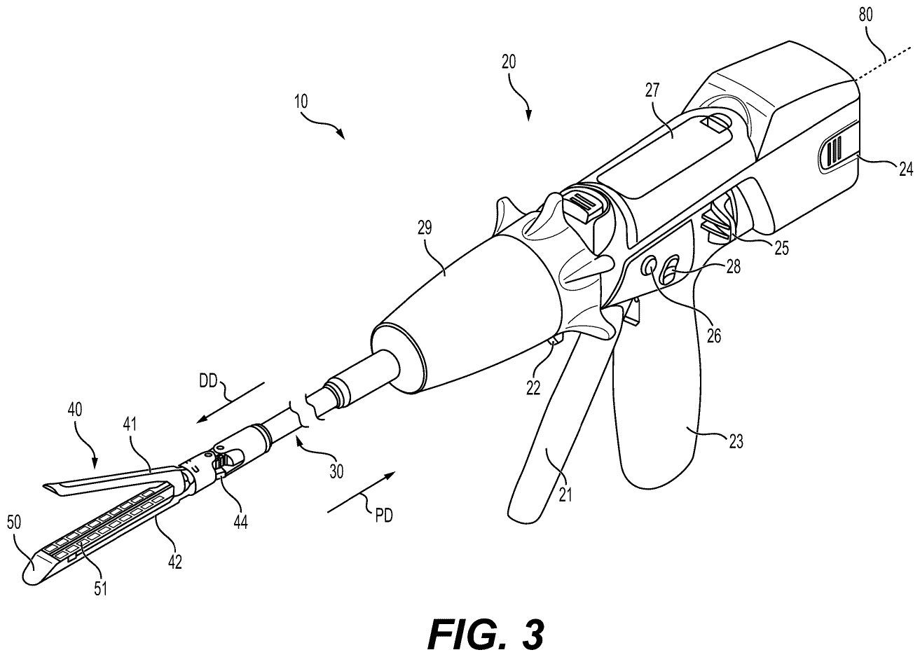

A motor-powered device drive system 60 is schematically shown in block diagram form in FIG. 1. In some examples, the drive system 60 is configured as an end effector drive system that performs powered actuation of an end effector 40 (FIG. 3), including clamping of tissue, and is discussed in greater detail with respect to FIG. 3. The drive system 60 is configured to selectively or simultaneously actuate a plurality of outputs, such as a first output 61A and a second output 61B.

The drive system 60 includes a motor control circuit 71 configured to drive a motor 62 (which functions as a motor input). The drive system 60 includes a transmission 63 configured to convert the rotational movement of a rotor of the motor 62 into actuation of one or more outputs 61A, 61B. The motor 62 and transmission 63 are collectively referred to herein as a motor assembly 72. The drive system 60 further includes a locking subsystem 73.

The motor control circuit 71 is illustrated as including a motor set circuit 64 (also referred to herein as a control circuit) and motor drive circuit 65 (also referred to herein as a motor control), which are illustrated as two separate blocks. The motor set circuit 64 and motor drive circuit 65 may be separate circuits or may be integrated as a single circuit. The motor set circuit 64 is configured to provide a motor setpoint signal output to the motor drive circuit 65. The motor setpoint signal is indicative of a target parameter, such as a target speed of a first output 61A or the second output 61B. The motor controller 65 is configured to provide a motor drive signal to the motor 62 such that the motor drive signal is based on the motor setpoint signal and intended to drive the motor 62 so that the first output 61A or the second output 61B is driven to the target parameter.

The motor set circuit 64 and the motor drive circuit 65 may include one or more processors and memory (e.g., one or more non-transitory computer-readable medium) with instructions that can be executed by the one or more processors to cause the motor set circuit 64 and the motor drive circuit 65 to drive the motor 62. The motor set circuit 64 and/or motor drive circuit 65 can include a feedback controller, which can be one of any feedback controllers, including, but not limited to a PID, a State Feedback, LQR, and/or an Adaptive controller, for example. The motor set circuit 64 and/or motor drive circuit 65 can include a power source to convert the signal from the feedback controller into a physical input such as a constant voltage, pulse width modulated (PWM) voltage, frequency modulated voltage, current, torque, and/or force, for example.

The motor drive circuit 65 is configured to electrically drive the motor 62 during at least a portion of a predetermined time period. The motor set circuit is configured to monitor a motor parameter of the motor during the predetermined time period. The motor set circuit 64 is configured to determine an end time of the predetermined time period based at least in part on the motor parameter. Additionally, or alternatively, the motor set circuit 64 is configured to determine the end time of the predetermined time period based on a parameter of the output. The drive system 60 can optionally include one or more sensor(s) (not shown) that can be configured to measure parameters of the outputs 61A, 61B directly.

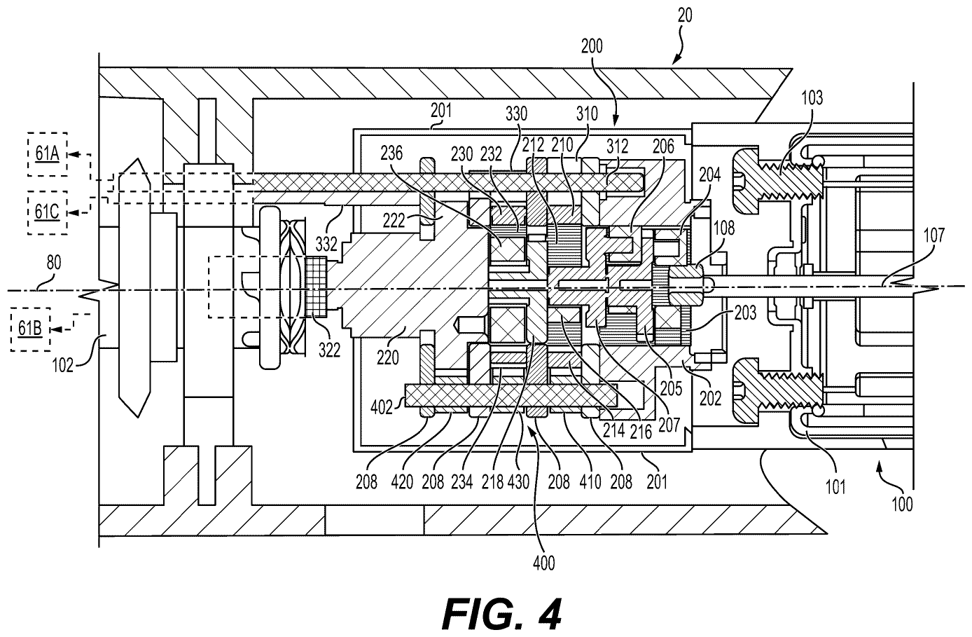

The transmission 63 in the presently disclosed technology is configured as a planetary gearbox 63 that is driven by the motor 62 and operably connects the motor 62 with the first output 61A and the second output 61B. As seen in FIG. 1, the planetary gearbox is comprised of a plurality of stages. In the present example, and for the purposes of description, the transmission 63 has two stages, such as a first stage that includes a first stage ring gear 63A and a second stage that includes a second stage ring gear 63B. Further details on exemplary configurations of the transmission 63 can be found in the description of FIGS. 4-8B. The first stage 63A engages with the first output 61A while the second stage 63B correspondingly engages with the second output 61B. It is noted that the numbering of these stages does not necessarily imply an order and/or indicate the stages are consecutive. For example, the second stage 63B in FIG. 1 (and as described herein) can have an intermediate stage interposed between it and the first stage 63A without departing from the spirit and scope of the present disclosure.

The locking subsystem 73 is selectively engageable (directly and/or indirectly) with at least one of the first stage ring gear 63A or the second stage ring gears 63A. In examples detailed herein, the locking subsystem 73 engages with the ring gears 63A, 63B of the respective stages, which is discussed in greater detail with respect to FIGS. 4-8B. The locking subsystem 73 includes a lock control 66 and a lock 67. The lock control 66 can take various forms, such as a lock control circuit, a mechanical control (e.g., a button, a lever, or a handle), or any other appropriate structure (and discussed in greater detail with respect to FIGS. 4-8B). Moreover, those skilled in the art will appreciate that, as used herein, the lock 67 depicted in FIG. 1 is not necessarily limited to a single lock, nor does it necessarily need to be a distinct component from the rest of the gearbox 63. For example, the lock 67 can comprise two or more locks or can include the ring gear lock inherent to the gearbox 63 without departing from the spirit and scope of the present disclosure. The lock 67 is controllable between an unlocked position, which allows the at least one of the first stage ring gear or the second stage ring gear to power at least one of the first output or the second output and a locked position that stops rotation of the at least one of the first stage ring gear or the second stage ring gear to lock at least one of the first output or the second output.

It is noted that the terms “unlocked position” and “locked position” encompass locks that move to mechanically (or otherwise) lock the ring gears and locks that do not necessarily move to lock the ring gear(s). Moreover, these terms are also used equivalently with the respective terms “unlocked state” and “locked state”. Use of the terms “unlocked” and “locked”, in the context of the gearbox 63, refer to the capability (or lack thereof) of the ring gear(s) 63A, 63B to rotate upon application of a driving force, which is discussed in greater detail below.

Additionally, it is noted that the lock 67 can be configured as a selective on/off control, which allows one of the ring gears to move while restraining the other. In a case where the motor is coupled to a ring gear and is capable of moving, it can also prevent it from moving by providing a second lock. For instance, if the motor 62 is coupled to an articulation control and the motor 62 moves, the articulation moves. However, there are two other motor states that are also important. If the motor 62 is not energized but coupled, it could be back driven, allowing for some mechanism motion of the articulation. If, however, the motor is dynamically braked (e.g., meaning the field effect transistors (FETs) for an H-bridge are configured to prevent electrical current through the motor), then the motor 62 resists back driving and the articulation is locked in place even if it is mechanically coupled to move. This is useful if the system is configured to allow the gearbox 63 to move, but the user is not actively requesting motor motion, which is then restraining the coupled system in its current location.

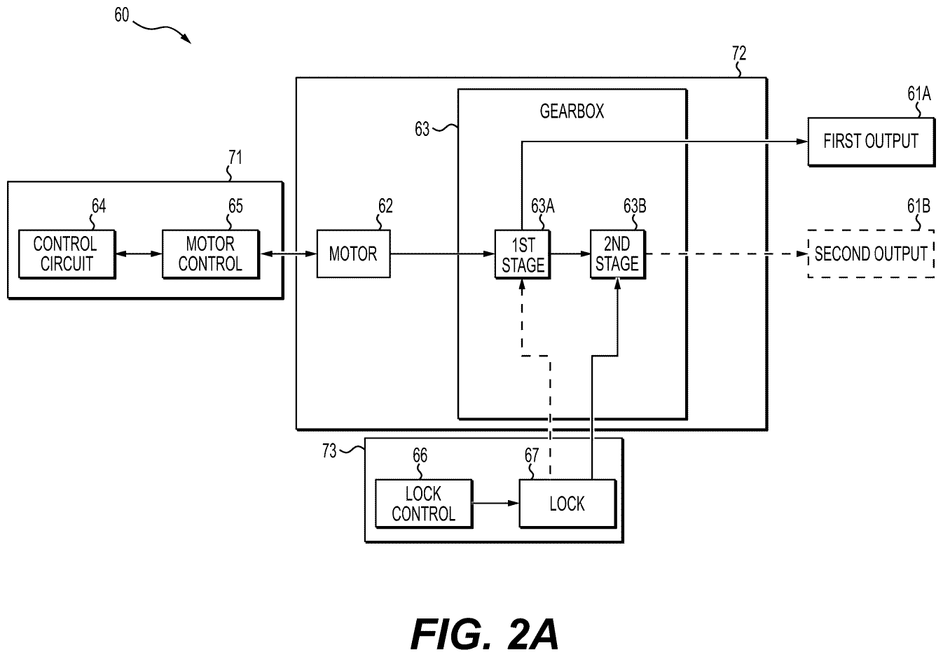

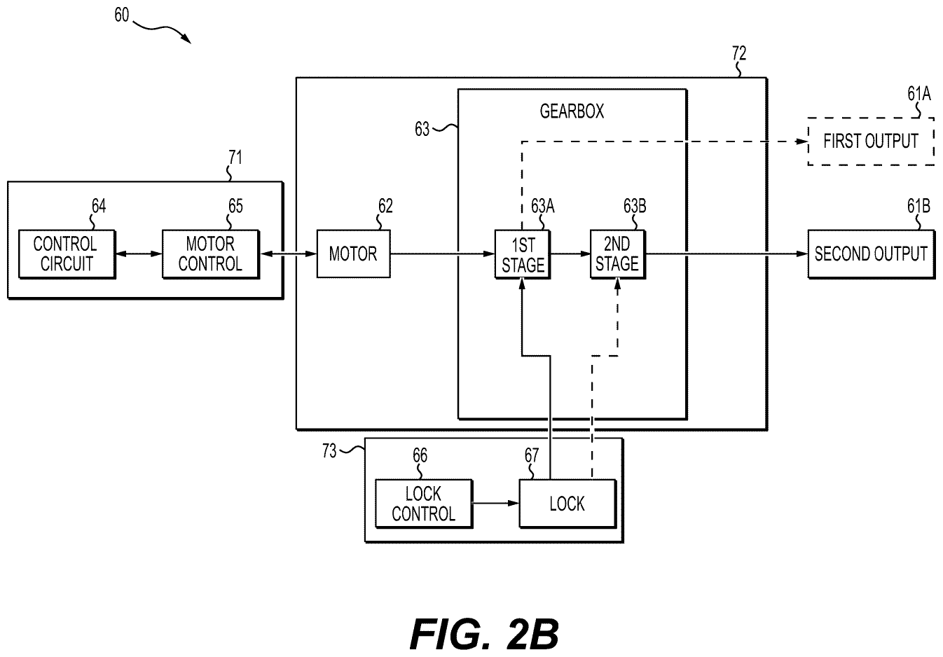

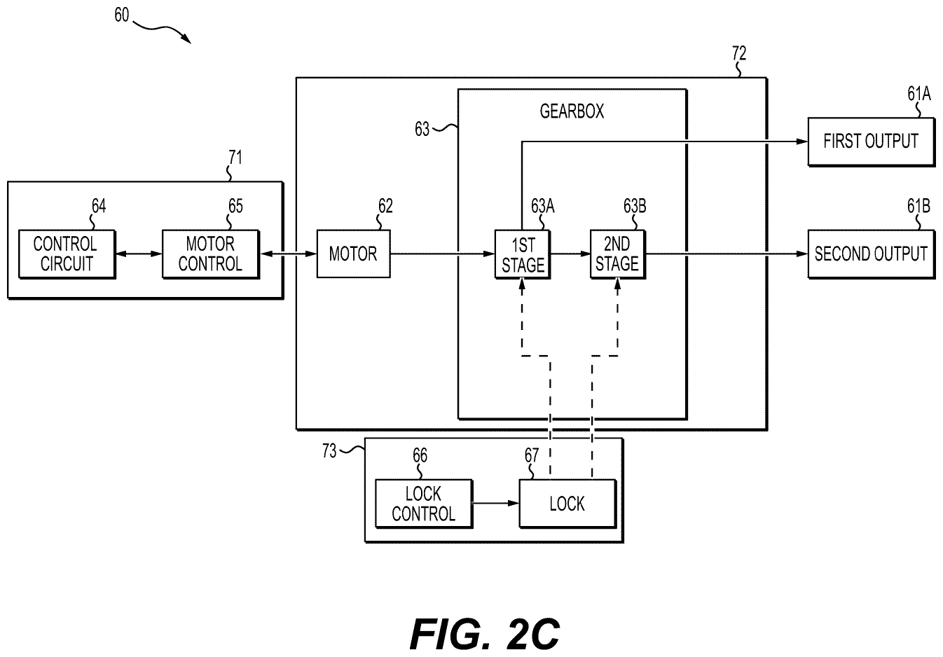

Reference is now made to FIGS. 2A-2C. FIGS. 2A-2C are block diagrams showing a plurality of different outputs 61A, 62B achievable by a single motor 62 due to the selective engagement of the lock 67 with one or more of the ring gears 63A, 63B of the respective stages. While two stages are shown in FIGS. 1-2C, it is noted that any number of stages and corresponding outputs can be employed in accordance with the following description without departing from the spirit and scope of the present disclosure. Additionally, it is noted that additional stages can be employed to achieve different gear ratios of the outputs, and do not necessarily need to be directly associated with a respective output.

FIG. 2A depicts a configuration of the drive system 60 where the lock control 66 controls the lock 67 such that the first stage ring gear 63A of the first stage of the transmission 63 is unlocked and free to rotate and the second stage ring gear 63B of the second stage of the transmission 63 is locked and prevented from rotation. In this configuration, upon driving of the motor 62, rotational movement of the rotor of the motor 62 is converted into actuation of the first output 61A by the transmission 63 via the first stage ring gear 63A. However, because the lock 67 prevents the second stage ring gear 63B from rotating, the second output 61B is not actuated.

FIG. 2B depicts a reverse configuration of the drive system 60 (relative to the one shown in FIG. 2B) where the lock control 66 controls the lock 67 such that the second stage ring gear 63B of the second stage of the transmission 63 is unlocked and free to rotate and the first stage ring gear 63A of the first stage of the transmission 63 is locked and prevented from rotation. In this configuration, upon driving of the motor 62, rotational movement of the rotor of the motor 62 is passed through the first stage and converted into actuation of the second output 61B by the transmission 63 via the second stage ring gear 63B. However, because the lock 67 prevents the first stage ring gear 63A from rotating, the first output 61A is not actuated.

FIG. 2C depicts a hybrid configuration of the drive system 60 where the lock control 66 controls the lock 67 such that both the first stage ring gear 63A and the second stage ring gear 63B of the second stage of the transmission 63 are unlocked and free to rotate. In this configuration, upon driving of the motor 62, rotational movement of the rotor of the motor 62 is simultaneously converted into actuation of the first output 61A via the first stage ring gear 63A and also passed through the first stage and converted into actuation of the second output 61B by the transmission 63 via the second stage ring gear 63B. In some examples, such as ones discussed below, the lock 67 can be designed such that one or more of the ring gears 63A, 63B can be, rather than fully locked or fully unlocked, partially locked, resulting in application of a braking force on the one or more ring gears 63A, 63B.

While FIGS. 1-2C provide an understanding of a general drive system 60 in which the transmission 63 of the present application can be employed, FIG. 3 provides a perspective view of an exemplary handheld powered surgical stapler 10 including a handle 20, a shaft 30, and an end effector 40 that includes outputs actuated by the aforementioned drive system 60. The handle 20 is configured to be grasped, manipulated, and actuated by a clinician. The shaft 30 is sized, shaped, and otherwise configured to extend through a body opening of the patient. The end effector 40 is configured deliver staples 51. The end effector 40 may also be configured to cut tissue within the body of the patient. As mentioned above, the drive system 60 discussed in reference to FIGS. 1-2C can be configured as an end effector drive system 60 that performs powered actuation of the end effector 40, including, but not limited to, clamping of tissue.

The shaft 30 is sized, shaped, and otherwise configured to extend through a body opening of the patient. The end effector 40 is configured deliver staples 51. The end effector 40 may also be configured to cut tissue within the body of the patient. The end effector 40 includes an anvil 41 and a staple jaw 42 (also referred to herein as the “channel”) opposite the anvil 41. The anvil 41 and channel 42 are collectively referred to herein as “jaws.” The channel 42 can include a staple cartridge 50 containing the staples 51. The staple cartridge 50 can be replaceable. Alternatively, the end effector 40 may be replaceable. The anvil 41 and channel 42 are illustrated in an open position. The anvil 41 and channel 42 can be moved toward each other to move the end effector 40 to a clamped configuration. For instance, tissue (not illustrated) can be positioned between the anvil 41 and channel 42 in the open position, and the anvil 41 can rotate toward the channel 42 to clamp the tissue. The end effector 40 can be actuated to deploy staples 51 into tissue during a firing stroke. Rotation of the anvil 41 to clamp tissue and deployment of staples 51 during a firing stroke are respectively motor driven by one or more motors (e.g., motor 100 of FIG. 4, not explicitly shown in FIG. 3). When the end effector 40 is in the clamped configuration, the firing trigger 22 can be pulled to cause deployment of staples 51 from the cartridge 50 and may also cause cutting of tissue.

Software to control tissue clamping by the end effector 40 can be stored in memory of the powered surgical stapler 10. The software can be configured to determine clamping time based on force and/or torque of motor(s) driving the end effector 40 during clamping. The software can be configured to provide user feedback and/or control operation of the surgical stapler based on the clamping time. For instance, the software can be configured to provide a visual display of clamping time (e.g. a count-down to the end of the clamping time period) so that the clinician is alerted when the tissue is sufficiently compressed to engage a firing stroke. Additionally, or alternatively, the software may lock-out the firing trigger 22 so that the clinician is unable to initiate the firing stroke until after the end of the clamping time period. Additionally, or alternatively, the software can be configured to automatically initiate a firing stroke at the end of the clamping time period. The surgical stapler 10 can include an articulation control circuit configured to modify the angle of the end effector 40 at the articulation joint 44 based at least in part on a motor parameter of the motor driving the end effector 40 during the clamping time period. The software can otherwise include instructions for operating the end effector 40 as described in greater detail elsewhere herein.

The surgical stapler 10 further includes an articulation joint 44 between the shaft 30 and the end effector 40. The articulation joint 44 is configured to permit the end effector 40 to be angled in relation to a longitudinal axis 80 of the shaft 30. As illustrated, the end effector 40, when oriented straight forward, is coaxial with the longitudinal axis 80. The articulation joint can be bent so that the end effector axis is angled toward a pitch axis, yaw axis, or some combination thereof. The articulation joint 44 can be bent manually by pressing the end effector 40 against tissue or other object or powered via one or more motor(s) of the surgical system. Additionally, or alternatively, the articulation joint 44 can be powered by the same or different motor configured to actuate clamping and/or firing of the end effector 40.

In preferred examples, two or more of these subsystems (e.g., clamping, articulation, and/or firing) can be powered by the same motor (illustrated in other figures, such as FIGS. 1 and 4). Further discussion on this configuration can be found in the examples of FIGS. 4-8B.

In some examples, the surgical stapler 10 can include an articulation control circuit configured to modify the angle of the end effector 40 based at least in part on a motor parameter of the motor driving the end effector 40 during the clamping time period. This can be advantageous in systems in which the end effector 40 is articulated by an articulation load. The articulation cable load can be improved during a firing stroke by either tightening or relaxing the cable through the articulation joint 44 depending on various conditions such as articulation joint stability (e.g., risk of de-articulation) and/or high cable load propagation.

The handle 20 can include a closure trigger 21, a firing trigger 22, and a grip 23 sized such that a clinician can single-handedly hold the surgical stapler 10 by the grip 23 while manipulating the closure trigger 21 or the firing trigger 22. The closure trigger 21 is operably connected to a motor (e.g., motor 100 of FIG. 4) disposed within the handle 20 such that when the closure trigger 21 is pulled, the motor is driven to cause the end effector 40 to clamp tissue. The firing trigger 22 is operably connected to a motor disposed within the handle 20 such that when the firing trigger 22 is pulled, the motor is driven to cause the end effector 40 to deploy staples 51 into the clamped tissue and may also cut the clamped tissue via a blade moving in a distal direction DD. The closure trigger 21 and the firing trigger 22 can be connected to separate respective motors, or the same motor 100 (FIG. 4, discussed in greater detail below).

The handle 20 can further include additional features such as a firing trigger lock mechanism (not illustrated) which can be manipulated to prevent actuation of the firing trigger 22, a power pack 24 configured to provide electrical power to the motor and other electrical components of the powered surgical stapler 10, a closure release button 25 which can be manipulated to release the end effector 40 and the closure trigger 21 from the clamped position, a home button 26 that can be pressed to cause the motor to move a firing assembly in the proximal direction PD to a home position, a manual override 27 including a mechanical actuator which can be manipulated to mechanically move the firing assembly proximally to the home position, articulation buttons 28 that can be pressed to cause a motor to articulate the end effector 40 at an articulation joint 44 so that the end effector 40 is at an angle with a longitudinal axis 80 of the shaft 30, a rotatable nozzle 29 configured to be rotated so that the shaft 30 and end effector rotate about the shaft axis 80, a display (not illustrated) configured to display information related to the surgical stapler, variations thereof, other compatible features of a powered surgical stapler handle, and combinations thereof.

Portions of the surgical stapler 10 may be detachable and interchangeable. Staples 51 may be housed in a staple cartridge 50 that is detachable from the end effector 40. The end effector 40 may be detachable from the shaft 30, and the shaft 30-handle 20 combination may be configured for use in connection with interchangeable end effectors. At least a portion of the shaft 30 including the end effector 40 may be detachable from the handle 20, and the handle 20 may be configured for use in connection with interchangeable shaft assemblies having different shaft lengths and/or different end effectors attached thereto.

The end effector drive system 60 can be configured to actuate the clamping assembly 61 to close the jaws 41, 42 of the end effector 40 illustrated in FIG. 3 and variations thereof as described herein and otherwise understood by a person skilled in the pertinent art. The end effector drive system 60 may further be configured to perform powered actuation of additional surgical operations of the end effector 40, such as driving a firing assembly to deploy staples 51 and pivoting the end effector 40 about an articulation joint 44.

In addition to handheld implementations, while not illustrated, the end effector drive system 60 can also be adapted for a robotic surgical system. As understood by a person skilled in the pertinent art, the physical location and physical structure of each of the components of the end effector drive system 60 have several suitable possibilities not listed herein for the sake of brevity.

Before turning to discussion of FIGS. 4-8B, it is noted that many of the following figures depict outputs (e.g., outputs 61A-61C) in dashed or solid line box form. These representations are merely intended to relay to the reader an understanding of their relative functional relationships with other components of the following examples, and are not intended to imply any particular structure and/or mechanical connections with specific components. In some of the following examples, the outputs 61A-61C are specifically referred to as, e.g. certain gears or elements. However, those skilled in the art will appreciate that the outputs 61A-61C can have different uses/structures than the ones described without departing from the spirit and scope of the present disclosure.

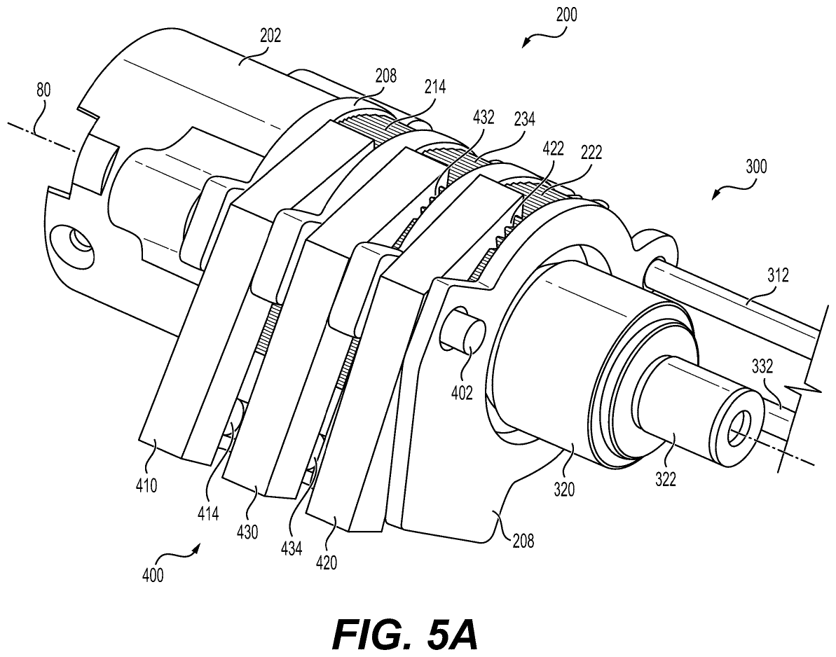

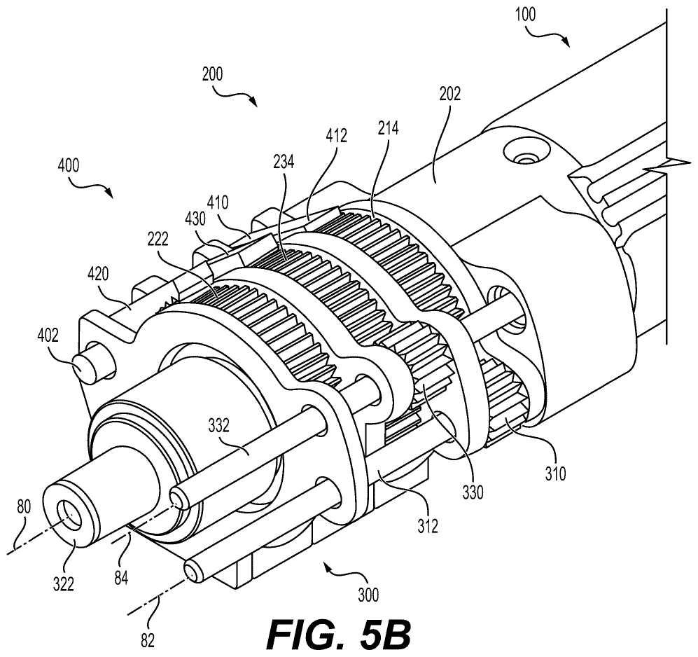

FIG. 4 is a partial cross-sectional view of a portion of the handle 20 of the surgical instrument/stapler 10. It is noted that the cross-sectional hatching in this and other figures is not indicative of a particular material; rather, different hatching styles are employed to better differentiate between components for the purposes of illustration. FIGS. 5A and 5B depict detail perspective views of the motor 100 and transmission 200. Making reference to FIG. 4, and as alluded to above, the handle 20 of the surgical stapler 10 of FIG. 3 can further include a motor 100 and a transmission 200, which operate equivalently to the aforementioned motor 62 and transmission 63. The motor 100 of the surgical instrument 10 can be operably coupled to a firing element 61B (also referred to herein as a second output and a firing control) and can drive (it is noted that the term power is also used synonymously with “power” in this context) the firing element 61B through the end effector 40 during a firing stroke. For example, the firing element 61B can cut tissue and/or fire staples into tissue during the firing stroke. A battery can supply current to the motor 100, for example, and the current supplied to the motor 100 can relate to the torque generated by the motor 100. Furthermore, the torque generated by the motor 100 can relate to the firing force exerted by the firing element 61B. The voltage across the motor can relate to the angular velocity of the motor 100, for example, which can relate to the speed of the firing element 61B.

In various examples, a control system (e.g., motor control circuit 71) in signal communication with the motor can supply current from the battery to the motor. In some embodiments, the control system can include speed management control, which can control the speed of the firing element, for example. The control system can include a variable resistance circuit and/or a voltage regulation circuit, for example, which can control the current supplied to and/or the voltage across the motor. In such embodiments, the control system can control the torque and/or the angular velocity of the motor, and thus, the firing force and/or the speed of the firing element coupled to the motor. For example, a voltage regulation circuit can regulate the voltage across the motor to affect the speed of the firing element.

In various examples, the control system can include a pulse width modulation circuit, and the control system can supply pulses of current to the motor.

A battery (not shown) can selectively supply current to the motor 100. The motor 100 can include a primary set of coils, and a secondary set of coils, for example. In various examples, the control system in signal communication with the motor 100 can selectively direct current to the primary set of coils and/or the secondary set of coils. For example, the control system 100 can supply current to the primary set of coils during a first operating state, and can supply current to the primary set of coils and the secondary set of coils during a second operating state, for example. In various examples, a switch (not shown) can move between an open position and a closed position to selectively supply current to the secondary set of coils, for example. In various examples, the sets of coils can be separately activatable.

In various examples, the motor 100 can generate a first amount of torque during the first operating state and a second amount of torque during the second operating state. The second amount of torque can be greater than the first amount of torque, for example. Furthermore, the additional torque generated by the secondary set of coils during the second operating state may prevent and/or limit lock-out of the firing element during a firing stroke. For example, the motor 100 can drive the firing element 61B distally during the first operating state and can drive the firing element 61B proximally during the second operating state. In various embodiments, the motor can generate greater torque when retracting the firing element 61B than when advancing the firing element. In such examples, retraction of the firing element may be improved. If the firing element becomes jammed, e.g., the tissue is too thick and/or tough for the firing element to cut and/or staple, the additional torque may be utilized to retract the firing element, for example.

In various examples, the motor 100 can be a brushed DC motor or a brushless DC motor, for example. In certain embodiments, the motor can be a stepper motor, such as a hybrid stepper motor, for example. Stepper motors can provide rotation control, such that an encoder is not necessary. Elimination of the encoder can reduce cost and/or complexity to the motor, for example. In some examples, the motor 100 can be a simplified stepper motor. For example, the motor 100 can comprise four electromagnetic poles spaced around the perimeter. In some examples, the motor 100 can be a hybrid stepper motor. The hybrid stepper motor can comprise permanent magnets and electromagnets, for example.

In various examples, the battery can have a volt-ampere limit or power threshold. In other words, the battery can supply a limited amount of energy per unit time. The power threshold of the battery can be related to the battery and/or circuit design. For example, thermal limits on the battery and/or the circuit, such as heat capacity and/or wire insulation, for example, can affect the power threshold. Furthermore, the power threshold of the battery can limit the amount of current supplied to the motor 100. In various examples, a motor utilizing speed management control, such as pulse width modulation, for example, may not require the maximum volt-amperes of the battery. For example, when the battery supplies current pulses at the maximum or optimized voltage to drive the firing element at the desired speed and maximum or optimized torque, surplus current may not be utilized to drive the firing element. In such examples, the surplus current can be used to produce additional torque. The motor 100 can include an additional or secondary set of coils, for example, and the surplus current can be selectively directed to the additional set of coils to generate additional torque. In such examples, the motor 100 can produce more torque at lower speeds, for example. In various examples, the control system can maximize the surplus current supplied to the secondary set of coils based on the volt-ampere limit of the battery, for example. Furthermore, in certain examples, the control system can optimize the torque generated by the motor during at least a portion of the firing stroke.

Some prior surgical instruments employ two separate motors. One motor is employed, for example, to advance the drive member distally through the loading unit which results in the closing of the anvil, cutting of tissue and firing of staples from the staple cartridge supported in the loading unit. The other motor is employed to articulate the loading unit about an articulation joint. The use of two motors in such devices may increase the complexity and add to the overall expense of the surgical instrument. For example, such arrangements may double the number of retraction systems and other mechanisms that could fail during use. The surgical instrument 10 depicted in, e.g., FIGS. 3-4 employs a single motor 100 which may be selectively employed to at least fire and articulate the surgical end effector 40, as well as for closure thereof, configured to perform at least one surgical procedure in response to firing motions applied thereto.

The surgical instrument 10 includes a handle/housing 20 that operably supports the motor 100 therein that is configured to generate rotary actuation motions. The motor 100 is operably coupled to a transmission 200 that has a selectively engageable lock 400 associated therewith which will be described in further detail below. The surgical instrument 10 may further include an articulation system that operably interfaces with the elongated shaft 30 for applying articulation motions to the surgical end effector 40 via the articulation joint 44.

The articulation mechanism may further include an articulation drive train arrangement that operably interfaces with a main articulation gear 61A (also referred to herein as the first output 61A and an articulation control) and the transmission 200. The articulation drive train arrangement can include an articulation drive shaft 312 (also referred to herein as an output shaft) extending along an articulation drive shaft axis 82 (which is parallel to the shaft axis 80) that is attached to an output of the transmission 200 as will be discussed in further detail below. A first articulation drive gear 310 is attached to the articulation drive shaft and is in meshing engagement with a central gear race (not shown) on a second articulation transfer gear (not shown) that is rotatably supported. Thus, rotation of the first articulation drive gear 310 results in rotation of the second central articulation transfer gear. A “third” articulation shaft gear (not shown) is mounted to a second articulation shaft (not shown) that has a “fourth” articulation worm gear (not shown) thereon. The third articulation shaft gear is in meshing engagement with the second central articulation transfer gear such that rotation of the first articulation drive gear ultimately results in the rotation of the third articulation shaft gear and the second articulation shaft. The fourth articulation worm gear is in meshing engagement with the main articulation gear such that rotation of the fourth articulation worm gear results in rotation of the main articulation drive gear 61A and ultimately application of articulation motions to the articulation link 44. As will be discussed in further detail below, the articulation drive shaft 312 is rotated by the motor 100 when the lock 400 is in an articulation control state.

As can be seen in FIG. 4, the motor 100 is operably coupled to the transmission 200. The transmission 200 may include a gearbox housing 201 that is coupled to the motor 100. For example, the gearbox housing 201 may be coupled to the motor housing 101 by screws 103 or other mechanical fasteners and/or fastener arrangements. The transmission may comprise a planetary gear arrangement contained within a ring gear housing 202 that is operably coupled to the motor shaft 107. In one arrangement for example, a ring gear 203 may be formed on the inner surface of the ring gear housing 202. A primary sun gear 108 is coupled to the motor shaft 107. The primary sun gear 108 is in meshing engagement with a plurality of first planetary gears 204 that are supported on a first planetary gear carrier 205 such that they are also in meshing engagement with the ring gear 203. A first sun gear (left side of planetary gear carrier 205) is formed on or otherwise attached to the first planetary gear carrier 205 and is in meshing engagement with a plurality of second planetary gears 206 that are supported on a second planetary gear carrier 207. The second planetary gears 206 are also supported in meshing engagement with the ring gear 203. A second sun gear (left side of second planetary gear carrier 207) is formed on or otherwise attached to the second planetary gear carrier 207 and is in meshing engagement with a plurality of third planetary gears 216. The third planetary gears 216 are supported on a third planetary gear carrier 218 and are supported in meshing engagement with inner teeth 212 of a first stage ring gear 210 (discussed in greater detail below). A third sun gear (left side of third planetary gear carrier 218) is formed on or is otherwise attached the third planetary gear carrier 218 and is in meshing engagement with a plurality of fourth planetary gears 236 that are supported by a third stage ring gear 220 and are supported in meshing engagement with inner teeth 232 of a second stage ring gear 230 (discussed in greater detail below). As seen in FIG. 4, the second stage ring gear 230 is interposed between the first stage ring gear 210 and the third stage ring gear 220.

It is noted that, for the purposes of explanation, the ring gears 210, 230, 220 are primarily described as first, second, and third stages as they are arranged sequentially. However, the numbering of the ring gears (and similar components) is non-sequential because, in some examples, and as mentioned elsewhere in the disclosure, the second stage 230 can be omitted. Thus, the second stage 230 can also be considered a third, optional stage of the transmission 200.

Moreover, a plurality of ring gear spacers 208 separate the respective stages of the transmission 200. These ring gear spacers 208 also support the articulation drive shaft 312 and a closure drive shaft 332 that extends along a closure drive shaft axis 84.

In the illustrated example, the surgical instrument 10 further includes a firing drive shaft 322 (also referred to herein as an output shaft) is connected to the third stage ring gear 220 at one side thereof, and operably coupled to a third output shaft 102 at an opposing side thereof. The third output shaft 102 connects with a third output 61B, such as a firing element as mentioned above.

Referring again to FIG. 4, a primary articulation drive gear 310 is attached to the articulation drive shaft 312 and is in meshing engagement with external teeth 214 (which radially oppose the plurality of inner teeth 212) of the first stage ring gear 210. Similarly, a primary closure drive gear 330 is attached to the closure drive shaft 332 (which is attached to, e.g., a main closure drive gear 61C) and is in meshing engagement with external teeth 234 (which radially oppose the inner teeth 232) of the second stage ring gear 230.

In various forms, the lock 400 includes a lock shaft 402 that rotatably supports a first lock 410 (also referred to herein as a locking pawl), a second lock 430, and a third lock 420 that, as seen particularly in FIGS. 5A and 5B, respectively engage the outer teeth 214 of the first stage ring gear 210, the outer teeth 222 of the third stage ring gear 220, and the outer teeth 234 of the second stage ring gear 230. In this present example, the locks 410, 420, 430 are configured as pivotable locking pawls that include teeth 412, 422, 432 that engage the respective outer teeth 214, 222, 234. These locks 410, 420, 430 can be biased to a locked position via respective springs 414, 424, 434 associated therewith. Moreover, the locks 410, 420, 430 can be operated in any appropriate manner (e.g., manually, electronically, etc.).

Operation of the lock 400 may be understood from reference to FIGS. 4-5B. When the first lock 410 is in an unlocked position (i.e., the first lock teeth 412 do not engage the outer teeth 214 of the first stage ring gear 210), the second lock 430 is in a locked position (i.e., the second lock teeth 432 engage the outer teeth 234 of the second stage ring gear 230), and the third lock 420 is in a locked position (i.e., the third lock teeth 422 engage the outer teeth 222 of the third stage ring gear 220), the first stage ring gear 210 is freely rotatable about the shaft axis 80 to drive the first articulation drive gear 310 to rotate about the articulation drive shaft axis 82 while the second stage ring gear 230 and the third stage ring gear 220 are prevented from rotation. Due to this configuration of the lock 400, only the main articulation gear 61A is driven by the motor 100 upon its rotation (via the interconnecting sun, ring, and planetary gears and carriers), while the firing element 61B and main closure drive gear 61C are effectively decoupled from the motor 100.

In another configuration, when the first lock 410 is in a locked position (i.e., the first lock teeth 412 engage the outer teeth 214 of the first stage ring gear 210), the second lock 430 is in an unlocked position (i.e., the second lock teeth 432 do not engage the outer teeth 234 of the second stage ring gear 230), and the third lock 420 is in a locked position (i.e., the third lock teeth 422 engage the outer teeth 222 of the third stage ring gear 220), the second stage ring gear 230 is freely rotatable about the shaft axis 80 to drive the primary closure drive gear 330 to rotate about the closure drive shaft axis 84 while the first stage ring gear 210 and the third stage ring gear 220 are prevented from rotation. Due to this configuration of the lock 400, only the main closure drive gear 61C is driven by the motor 100 upon its rotation (via the interconnecting sun, ring, and planetary gears and carriers), while the main articulation gear 61A and firing element 61B are effectively decoupled from the motor 100.

In yet another configuration, when the first lock 410 is in a locked position (i.e., the first lock teeth 412 engage the outer teeth 214 of the first stage ring gear 210), the second lock 430 is in an locked position (i.e., the second lock teeth 432 engage the outer teeth 234 of the second stage ring gear 230), and the third lock 420 is in an unlocked position (i.e., the third lock teeth 422 do not engage the outer teeth 222 of the third stage ring gear 220), the third stage ring gear 220 is freely rotatable about the shaft axis 80 to drive the primary closure drive gear 330 to rotate about the closure drive shaft axis 84 while the first stage ring gear 210 and the second stage ring gear 230 are prevented from rotation. Due to this configuration of the lock 400, only the firing element 61B is driven by the motor 100 upon its rotation (via the interconnecting sun, ring, and planetary gears and carriers), while the main articulation gear 61A and main closure drive gear 61C are effectively decoupled from the motor 100.

Of course, those skilled in the art will appreciate that, in addition to the above-described configurations, additional locking/unlocking combinations are possible without departing from the spirit and scope of the present disclosure. For example, rather than only one lock 410, 420, 430 being unlocked, two or all three locks 410, 420, 430 could be set to the unlocked position, resulting in the motor 100 simultaneously driving two or more of the outputs 61A-61C. Moreover, as mentioned above, while three stages and outputs are shown and described that are controlled by a single motor 100, this number can vary without departing from the spirit and scope of the present disclosure. For instance, the second stage in the present example could be omitted such that the third stage ring gear 220 operates as a second stage ring gear 220 in this system. Alternatively, the transmission 200 can include additional stages to drive additional outputs. When employed in a surgical devices, these outputs include, but are not limited to, firing, closing, articulation, proximal shaft rotation, and/or distal head rotation.

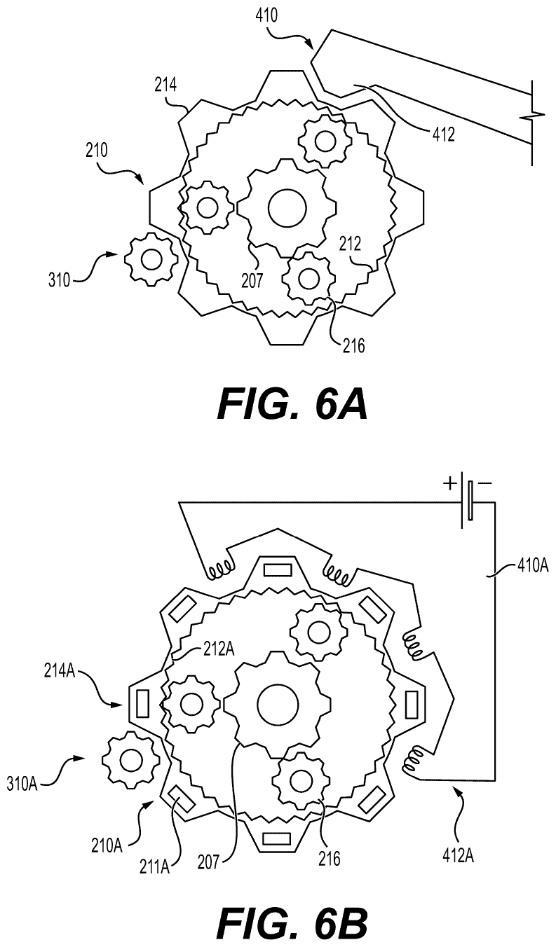

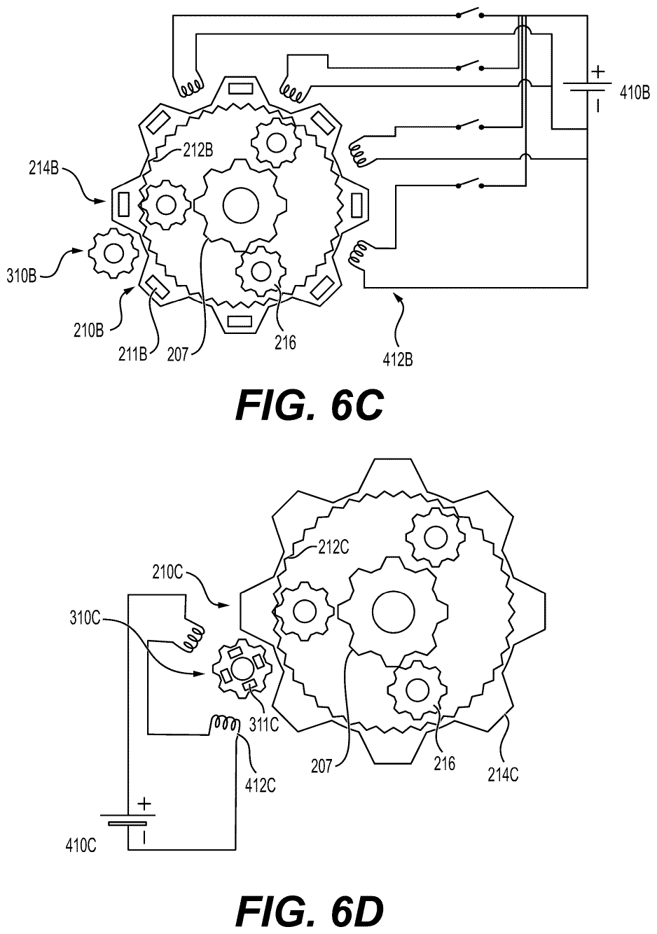

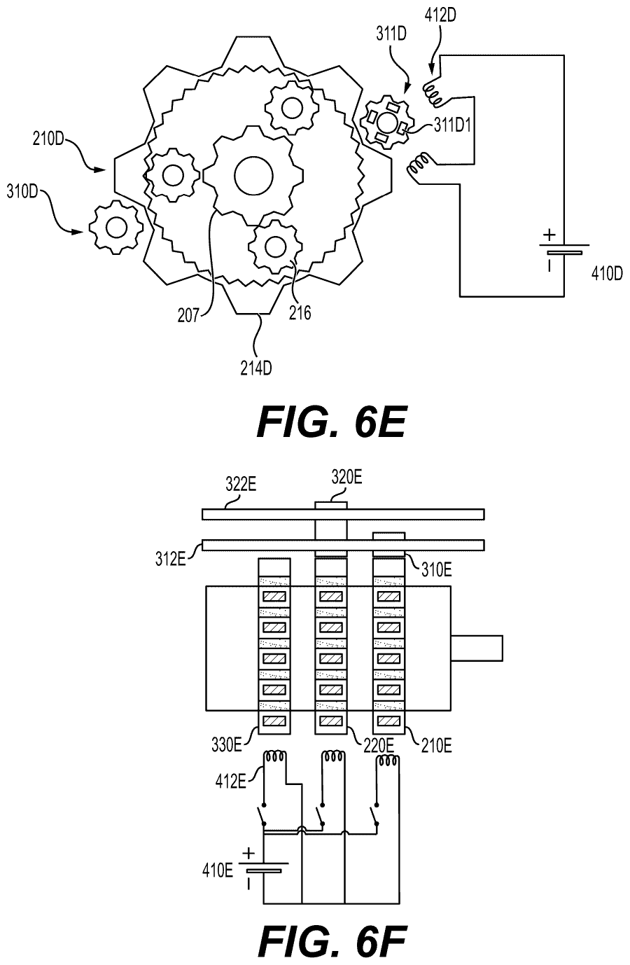

FIG. 6A is a schematic pictorial illustration of a detail elevation view of one of the stages and locks of the example described in FIGS. 4-5B, with FIGS. 6B-6F depicting alternative configurations thereof. It is noted that the figures are illustrative in nature to show potential layouts and functional relationships between components of the presently disclosed technology, and are not necessarily accurate to the exact physical structure of an exemplary system. Moreover, it is noted that this discussion applies to other stages of the example of FIGS. 4-5B, such as the second stage.

As seen in FIG. 6A, the sun gear of the second planetary gear carrier 207 meshes with the third planetary gears 216. The inner teeth 212 of first stage ring gear 210 also mesh with the third planetary gears 216. Thus, when the tooth or teeth 412 of the first lock 410 engage the outer teeth 214 of the first stage ring gear 210, the third planetary gears 216 orbit around the sun gear but the ring gear 210 cannot rotate. When the tooth or teeth 412 of the first lock 410 does not engage the outer teeth 214 of the first stage ring gear 210, the third planetary gears 216 orbit around the sun gear and also drive the first stage ring gear 210 to rotate, causing the primary articulation drive gear 310 to rotate with the outer teeth 214 it is meshed with.

As mentioned above, FIGS. 6B-6F depict variants of the lock 400. In these examples, a mechanical lock 400 is replaced with some form of an electromagnetic lock that can be used to induce resistance in the non-active stages in the transmission 200.

As seen in FIG. 6B, the sun gear of the second planetary gear carrier 207 meshes with the third planetary gears 216. The inner teeth 212A of a modified first stage ring gear 210A also mesh with the third planetary gears 216. In this example, the modified first stage ring gear 210A includes one or more embedded magnets 211A, and the lock 410A includes one or more electromagnetic coils 412A and/or inductors to produce an electromagnetic resistive force with a control circuit (rather than a mechanical lock). Thus, when current is run through the electromagnetic coil(s) 412A of the first lock 410A (referred to herein as a locked position or state), the magnet(s) 211A of the first stage ring gear 210A and the electromagnetic coil(s) 412A induce electromagnetic forces to either prevent the ring gear 210A from rotating or reduce the speed that it is able to rotate (depending on the magnitude of the electromagnetic forces induced). When the electromagnetic coil(s) 412A are deactivated (referred to herein as an unlocked position or state), the third planetary gears 216 orbit around the sun gear and also drive the first stage ring gear 210A to rotate, causing the primary articulation drive gear 310A to rotate with the outer teeth 214A it is meshed with.

As seen in FIG. 6C, the sun gear of the second planetary gear carrier 207 meshes with the third planetary gears 216. The inner teeth 212B of a modified first stage ring gear 210B also mesh with the third planetary gears 216. In this example, like the example of FIG. 6B, the modified first stage ring gear 210B includes one or more embedded magnets 211B, and the lock 410B includes a plurality of independently controllable electromagnetic coils 412B and/or inductors to produce an electromagnetic resistive force with a control circuit for finer precision/control of the first stage ring gear 210B. Thus, when current is run through one or more of the electromagnetic coil(s) 412B of the first lock 410B (referred to herein as a locked position or state), the magnet(s) 211B of the first stage ring gear 210B and the electromagnetic coil(s) 412B induce electromagnetic forces to either prevent the ring gear 210B from rotating or reduce the speed that it is able to rotate (depending on the magnitude of the electromagnetic forces induced). When the electromagnetic coil(s) 412B are deactivated (referred to herein as an unlocked position or state), the third planetary gears 216 orbit around the sun gear and also drive the first stage ring gear 210B to rotate, causing the primary articulation drive gear 310B to rotate with the outer teeth 214B it is meshed with.

As seen in FIG. 6D, another configuration of the locking system is shown. In this example, the sun gear of the second planetary gear carrier 207 meshes with the third planetary gears 216. The inner teeth 212C of a first stage ring gear 210C also mesh with the third planetary gears 216. In this example, a modified primary articulation drive gear 310C includes one or more embedded magnets 311C, and the lock 410C includes one or more independently controllable electromagnetic coils 412C and/or inductors to produce an electromagnetic resistive force with a control circuit. Thus, when current is run through one or more of the electromagnetic coil(s) 412C of the first lock 410C (referred to herein as a locked position or state), the magnet(s) 311C and the electromagnetic coil(s) 412C induce electromagnetic forces to either prevent the primary articulation drive gear 310C from rotating or reduce the speed that it is able to rotate (depending on the magnitude of the electromagnetic forces induced). When the electromagnetic coil(s) 412C are deactivated (referred to herein as an unlocked position or state), the third planetary gears 216 orbit around the sun gear and also drive the first stage ring gear 210C to rotate, causing the primary articulation drive gear 310C to rotate with the outer teeth 214C it is meshed with.

As seen in FIG. 6E, another configuration of the locking system is shown. In this example, the sun gear of the second planetary gear carrier 207 meshes with the third planetary gears 216. The inner teeth 212D of a first stage ring gear 210D also mesh with the third planetary gears 216. In this example, separate spur gear 311D is provided that includes one or more embedded magnets 311D1, and the lock 410D includes one or more independently controllable electromagnetic coils 412D and/or inductors to produce an electromagnetic resistive force with a control circuit. In this example, the spur gear 311D, similar to the primary articulation drive gear 310D, meshes with the outer teeth 214D of the first stage ring gear 210D. Thus, when current is run through one or more of the electromagnetic coil(s) 412D of the first lock 410D (referred to herein as a locked position or state), the magnet(s) 311D1 and the electromagnetic coil(s) 412D induce electromagnetic forces to either prevent the spur gear 311D from rotating or reduce the speed that it is able to rotate (depending on the magnitude of the electromagnetic forces induced). When the electromagnetic coil(s) 412C are deactivated (referred to herein as an unlocked position or state), the third planetary gears 216 orbit around the sun gear and also drive the first stage ring gear 210D to rotate, causing the primary articulation drive gear 310D to rotate with the outer teeth 214D it is meshed with (since the spur gear 311D does not prevent or reduce the ability to rotate).

As seen in FIG. 6F, another configuration of the locking system is shown, which demonstrates how different levels of electromagnetic power (for locking, as discussed above) can be applied to different stages of the transmission 200. In this example, similar to the examples detailed above, respective first, second, and third stage gears 210E, 220E, 230E drive respective spur gears (e.g., output gears 310E, 320E with respective output shafts 312E, 322E). The gears 210E, 220E, 230E include magnets that interact with electromagnetic coils 412E of another alternative lock 410E. In the different stages that have different outputs with different purposes, this allows different levels of power to be applied to the different stages to achieve varied locking/braking forces. By way of example, some electromagnetic force can be applied to the closure stage of the transmission, while the stage associated with firing allows the full force of the motor to drive it. In short, this configuration allows for precise turning of friction/braking for the different outputs, and can also be used for load and/or balancing of the gearset.

It is noted that the above examples of FIGS. 6B-6F primarily describe the electromagnetic forces as contributing to the resistance to rotating (i.e., providing a locking/braking force), but these systems can also be use in an additive manner. In other words, the locks 410A-410E can also be used help rotate the respective stage ring gears by reducing the force required for the input motor 100 to rotate them. This can be achieved, for example, by the coils repelling the magnets.

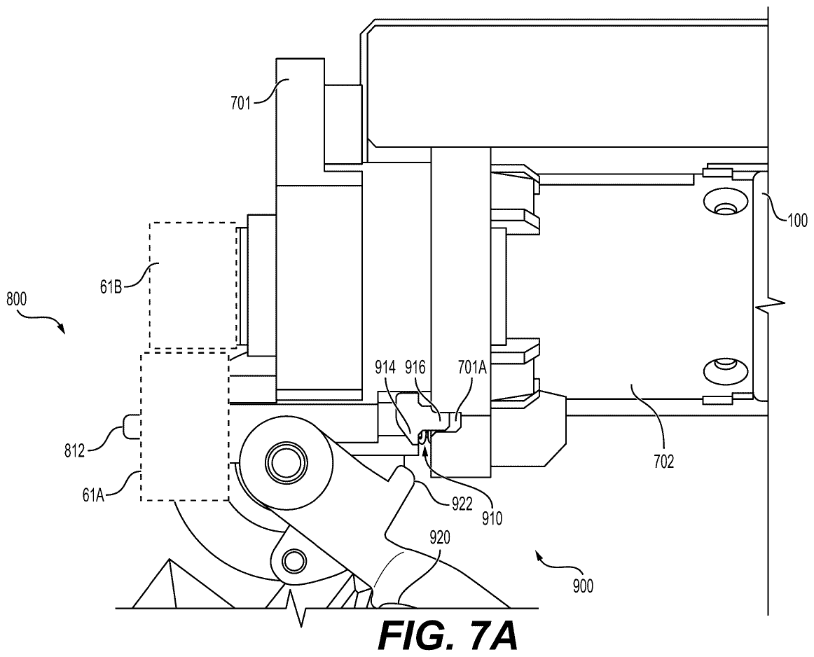

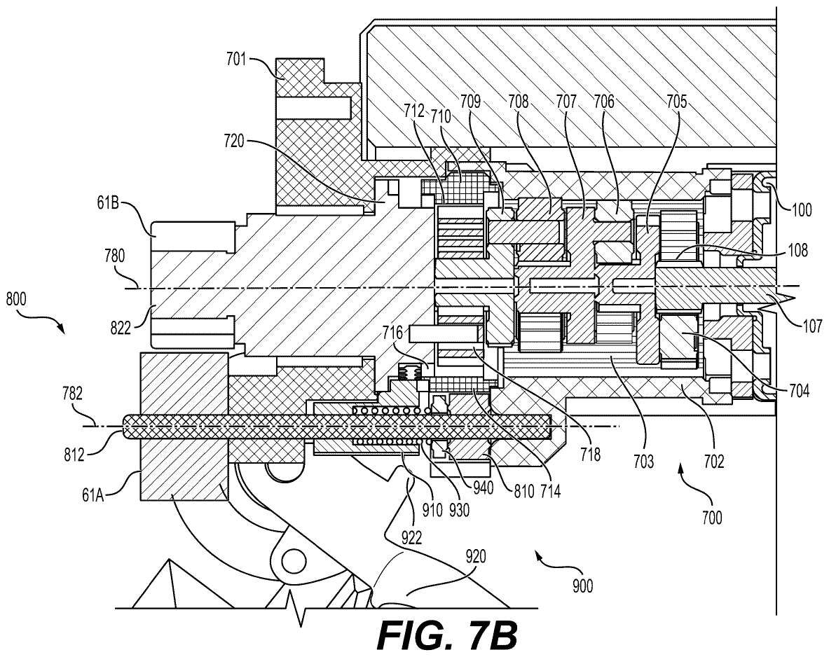

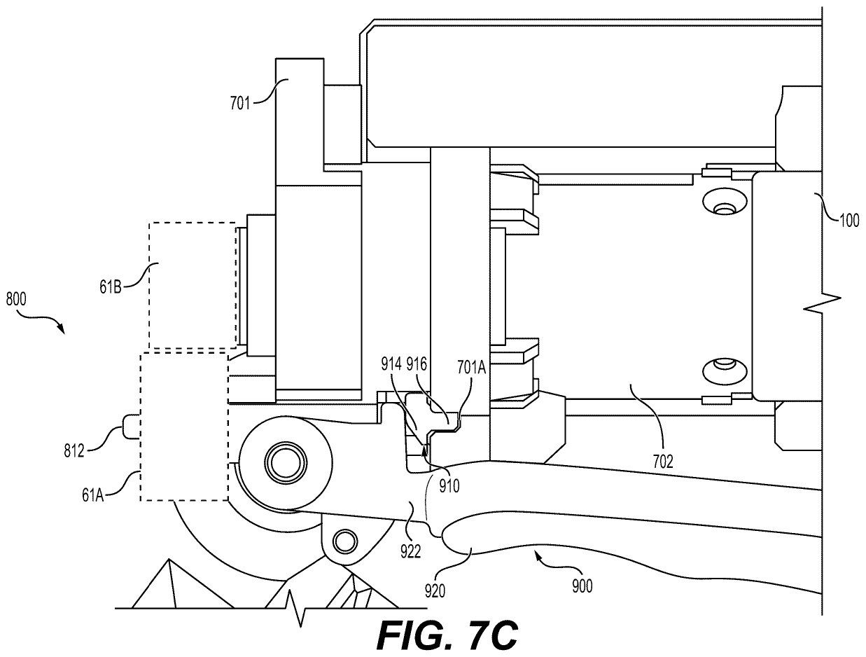

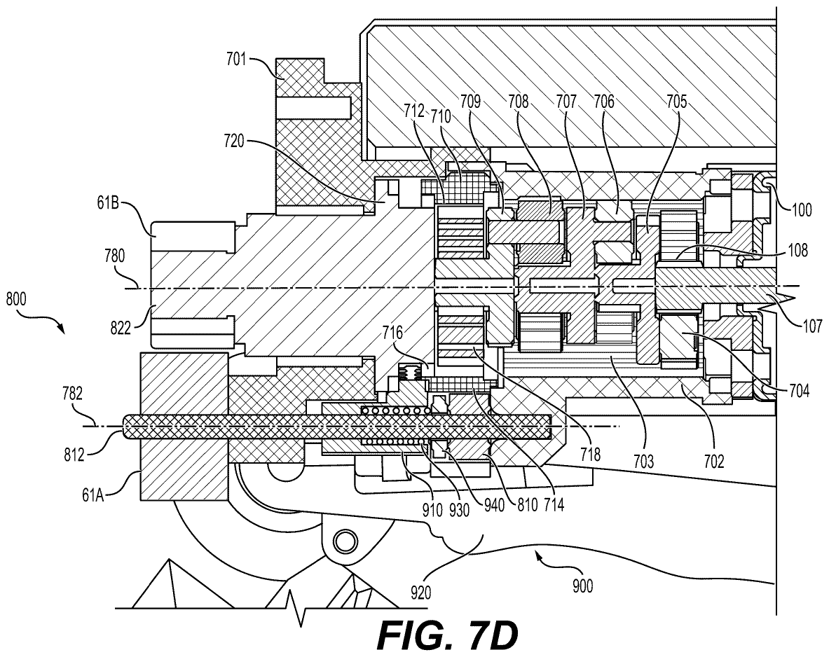

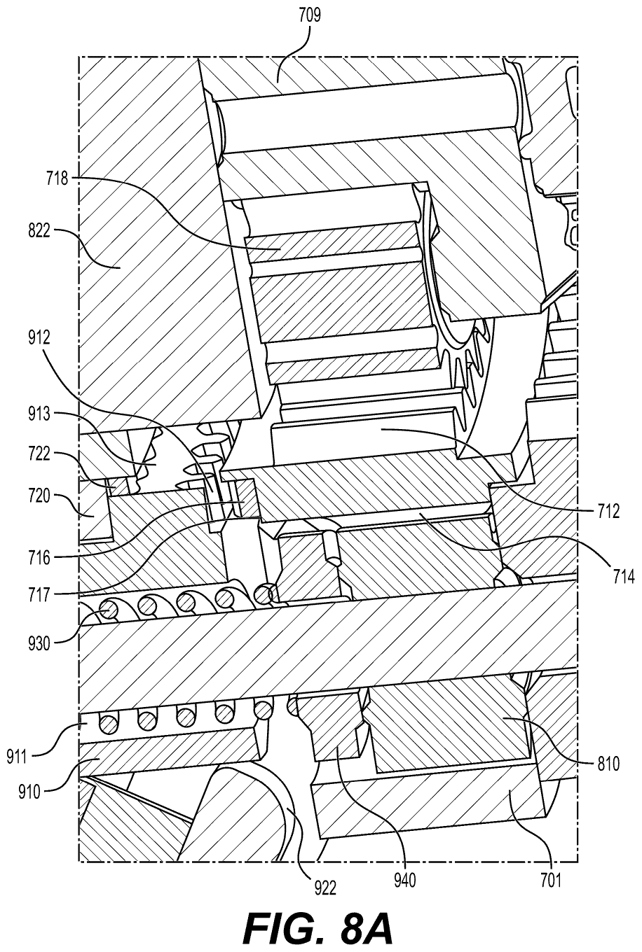

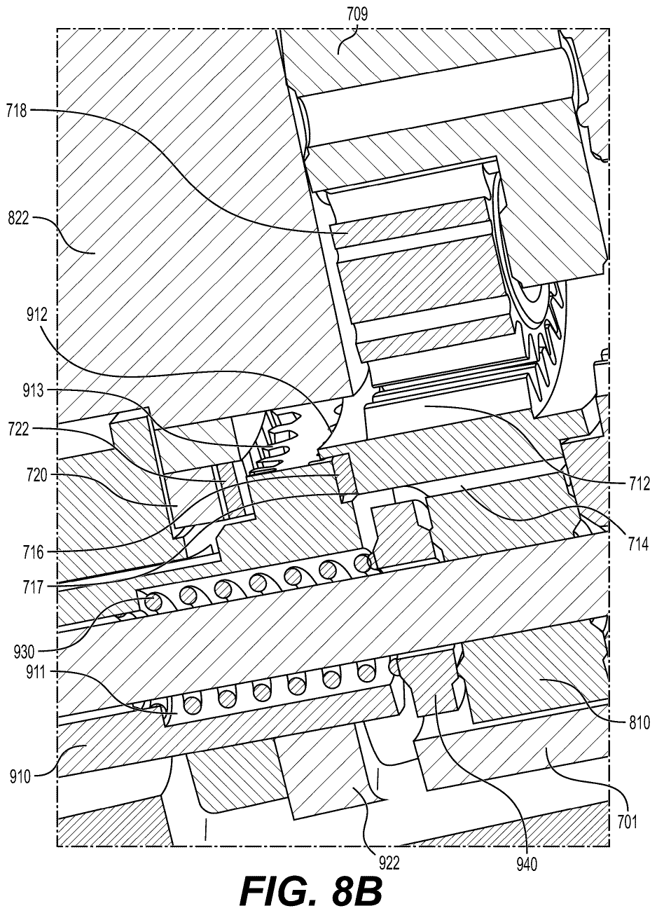

FIGS. 7A-8B depict yet another configuration of transmission and lock assembly in accordance with the present disclosure. This example is similarly configured to achieve the same functional goals as that of the previously described examples, which is to provide a transmission that includes stages that are selectively lockable/unlockable in order to drive different outputs.

Making reference to FIG. 7A, and as alluded to above, a system in accordance with the present disclosure can include a motor 100 and a transmission 700, which operate equivalently to the aforementioned motor 62 and transmission 63. The motor 100 can be employed with the surgical instrument 10 (or other appropriate device), which is operably coupled to a plurality of outputs 800, which include second output 61B (such as a portion of a firing element), and can drive the firing element through the end effector 40 (FIG. 3) during a firing stroke. For example, the firing element can cut tissue and/or fire staples into tissue during the firing stroke. The motor 100 can be designed, powered, and controlled as previously described; therefore, further discussion on the motor in this example is omitted for brevity.

The surgical instrument 10 includes a handle/housing 20 that operably supports the motor 100 therein that is configured to generate rotary actuation motions. The motor 100 is operably coupled to a transmission 700 that has a selectively engageable lock assembly 900 associated therewith which will be described in further detail below. The surgical instrument 10 may further include an articulation system that operably interfaces with the elongated shaft 30 for applying articulation motions to the surgical end effector 40 via the articulation joint 44, as previously discussed.

The articulation mechanism may further include an articulation drive train arrangement that operably interfaces with a first output 61A, that connects with a main articulation gear, and the transmission 700. The articulation drive train arrangement can include an articulation drive shaft 812 extending along an articulation drive shaft axis 782 (FIG. 7B) that is attached to an output of the transmission 700 as will be discussed in further detail below. A first articulation drive gear 810 is attached to the articulation drive shaft 812 and is in meshing engagement with a central gear race (not shown) on a second articulation transfer gear (not shown) that is rotatably supported. Thus, rotation of the first articulation drive gear 810 results in rotation of the second central articulation transfer gear. A “third” articulation shaft gear (not shown) is mounted to a second articulation shaft (not shown) that has a “fourth” articulation worm gear (not shown) thereon. The third articulation shaft gear is in meshing engagement with the second central articulation transfer gear such that rotation of the first articulation drive gear ultimately results in the rotation of the third articulation shaft gear and the second articulation shaft. The fourth articulation worm gear is in meshing engagement with the main articulation gear such that rotation of the fourth articulation worm gear results in rotation of the main articulation drive gear 61A and ultimately application of articulation motions to the articulation link 44. As will be discussed in further detail below, the articulation drive shaft 812 is rotated by the motor 100 when the lock 900 is in an articulation control state (discussed in greater detail below).

As can be seen in FIG. 7B, the motor 100 is operably coupled to the transmission 700. The transmission 700 may include a gearbox housing 701 that is coupled to the motor 100. For example, the gearbox housing 701 may be coupled to the motor housing by screws or other mechanical fasteners and/or fastener arrangements. The transmission may comprise a planetary gear arrangement contained within a ring gear housing 702 that is operably coupled to the motor shaft 107. In one arrangement for example, a ring gear 703 may be formed on the inner surface of the ring gear housing 702. A primary sun gear 108 is coupled to the motor shaft 107. The primary sun gear 108 is in meshing engagement with a plurality of first planetary gears 704 that are supported on a first planetary gear carrier 705 such that they are also in meshing engagement with the ring gear 703. A first sun gear (left side of planetary gear carrier 705 in FIG. 7B) is formed on or otherwise attached to the first planetary gear carrier 705 and is in meshing engagement with a plurality of second planetary gears 706 that are supported on a second planetary gear carrier 707. The second planetary gears 706 are also supported in meshing engagement with the ring gear 703. A second sun gear (left side of second planetary gear carrier 707) is formed on or otherwise attached to the second planetary gear carrier 707 and is in meshing engagement with a plurality of third planetary gears 708. The third planetary gears 708 are supported on a third planetary gear carrier 710 and are supported in meshing engagement with the ring gear 703. A third sun gear (left side of third planetary gear carrier 710) is formed on or is otherwise attached to the third planetary gear carrier 710. Moreover, the third sun gear is in meshing engagement with a plurality of fourth planetary gears 718 that are supported on an output shaft 822 and are supported in meshing engagement with inner teeth 712 of a first stage ring gear 710. The first stage ring gear 710 can include a first set of outer teeth 714 (extending radially outwards) and a second set of outer teeth 716 (extending from a lateral side 717 of the first stage ring gear 710) that are oriented perpendicularly relative to one another (see, e.g., FIG. 8A). A second stage ring gear 720 can be attached to or otherwise integral with the output shaft 822 and includes a first set of outer teeth 722 that extend towards and parallel with the second set of outer teeth 716 of the first stage ring gear 710. In the illustrated example, the output shaft 822 can be configured as a firing drive shaft 822. A primary articulation drive gear 810 is attached to the articulation drive shaft 812 and is in meshing engagement with the first set of external teeth 714 of the first stage ring gear 210.

In various forms, the lock assembly 900 includes a locking pawl 910 (also referred to herein as a lock), a handle 920 (or other control), a spring 930 (e.g., a coil spring or other appropriate biasing element), and a spring retainer 940.

The locking pawl 910 includes a first set of teeth 912 oriented parallel to the articulation drive shaft axis 782 in a first direction and a second set of teeth 913 oriented parallel to the articulation drive shaft axis 782 in the opposite direction. The locking pawl 910 defines a cavity 911 (FIG. 8A) for receiving the spring 930, with the spring retainer 940 preventing the spring 930 from exiting the cavity 911. Moreover, the locking pawl 910 is mounted on and is translatable along the articulation drive shaft 812 to selectively engage with the first stage ring gear 710 and the second stage ring gear 720. As seen best in FIG. 7A, the locking pawl 910 further includes a cam surface 914 and an axial guide 916 that rides in a channel 701A defined in the transmission housing 701. The handle 920 includes an arcuate engagement surface 922 that is configured to engage the cam surface 914, and is discussed in greater detail below.

Operation of the lock assembly 900 may be understood from reference to 7A-8B.

When the handle 920 is in a first, open position (FIGS. 7A-7B and 8A) the locking pawl 910 is biased by the spring 930 such that the second set of teeth 913 of the locking pawl 910 mesh with the first set of outer teeth 722 of the second stage ring gear 720 (FIG. 8A). In this second stage locked position, the second stage ring gear 720 (and, thus, also the output shaft 822) is prevented from rotating while the first stage ring gear 710 is freely rotatable. Thus, in this position, only the main articulation gear 61A is driven by the motor 100 upon its rotation (via the interconnecting sun, ring, and planetary gears and carriers), while the firing element 61B is effectively decoupled from the motor 100.

When the handle 920 is rotated or otherwise moved to a second, closed position (FIGS. 7C-7D and 8B), the engagement surface 922 of the handle 920 rides on the cam surface 914 to drive the locking pawl 910 axially out of engagement with the second stage ring gear 720 and into engagement with the first stage ring gear 710 such that the second set of teeth 716 of the first stage ring gear 710 mesh with the first set of teeth 912 of the locking pawl 910 (FIG. 8B). In this first stage locked position, the first stage ring gear 710 (and, thus, also the output shaft 812) is prevented from rotating while the second stage ring gear 720 is freely rotatable. Thus, in this position, only the firing element 61B is driven by the motor 100 upon its rotation (via the interconnecting sun, ring, and planetary gears and carriers), while the main articulation gear 61A is effectively decoupled from the motor 100.

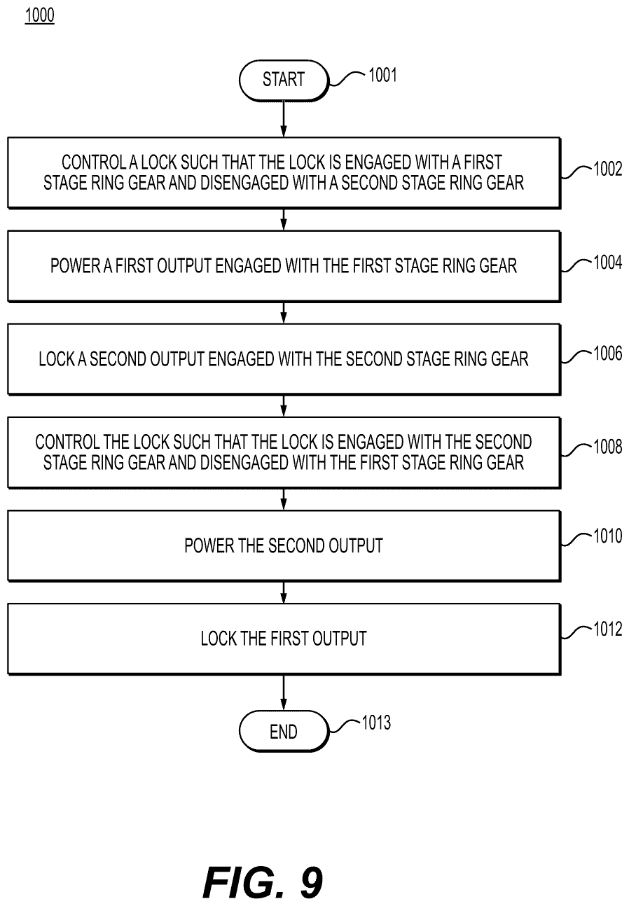

Making reference to FIG. 9, a method 1000 of operating a motor-powered device can include the following steps. A lock is controlled 1002 such that the lock is engaged with a first stage ring gear and disengaged with a second stage ring gear. This resulted in a first output being powered 1004 that is engaged with the first stage ring gear, and a second output being locked 1006 that is engaged with the second stage ring gear. The lock is controlled 1008 such that the lock is engaged with the second stage ring gear and disengaged with the first stage ring gear. This results in the second output being powered 1010 and the first output being locked. In some examples, the method can further include controlling the lock such that the lock is disengaged with the second stage ring gear and disengaged with the first stage ring gear, as well as powering the first output and the second output simultaneously.

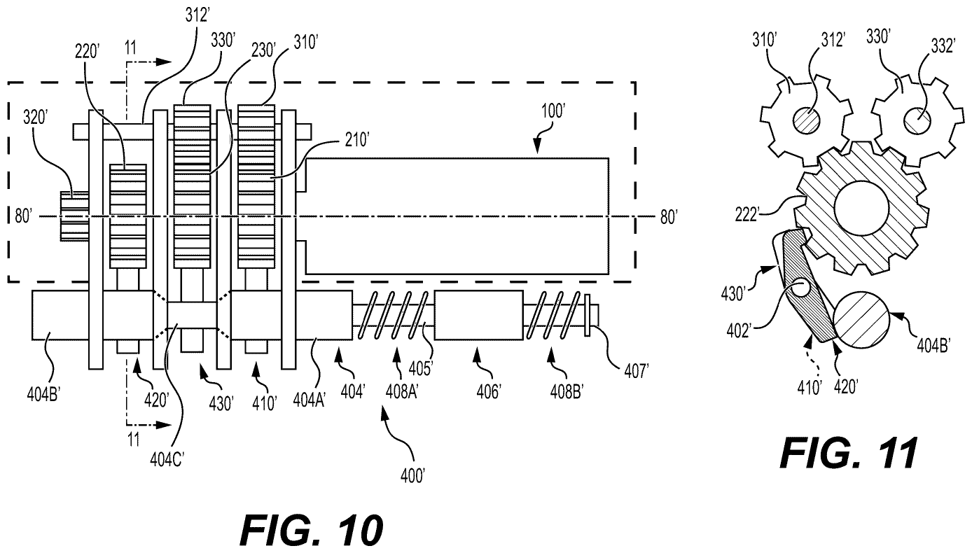

FIGS. 10-14 depict yet another configuration of transmission and lock assembly in accordance with the present disclosure. This example is similarly configured to achieve the same functional goals as that of the previously described example of FIG. 3-5B, which is to provide a transmission that includes stages that are selectively lockable/unlockable in order to drive different outputs.

Making reference to FIG. 10, and as alluded to above, a system in accordance with the present disclosure can include a motor 100′ and a transmission 200′, which operate equivalently to the aforementioned motor 62, 100 and transmission 63, 200. The motor 100′ can be employed with the surgical instrument 10 (or other appropriate device), which is operably coupled to a plurality of outputs, which include second output 61B (such as a portion of a firing element), and can drive the firing element through the end effector 40 (FIG. 3) during a firing stroke. For example, the firing element can cut tissue and/or fire staples into tissue during the firing stroke. The surgical instrument 10 may further include an articulation system that operably interfaces with the elongated shaft 30 for applying articulation motions to the surgical end effector 40 via the articulation joint 44, as previously discussed. The motor 100′ can be designed, powered, and controlled as the motors previously described; therefore, further discussion on the motor in this example is omitted for brevity.

As discussed in previous examples, the surgical instrument 10 includes a handle/housing 20 that operably supports the motor 100′ therein that is configured to generate rotary actuation motions. The motor 100′ is operably coupled to a transmission 200′ that has a selectively engageable lock 400′ associated therewith. The surgical instrument 10 may further include an articulation system that operably interfaces with the elongated shaft 30 for applying articulation motions to the surgical end effector 40 via the articulation joint 44.

Like the example described with respect to FIG. 4, the articulation mechanism may further include an articulation drive train arrangement that operably interfaces with a main articulation gear 61A (depicted in FIG. 4) and the transmission 200′. The articulation drive train arrangement can include an articulation drive shaft 312′ (also referred to herein as an output shaft) that is attached to an output of the transmission 200′. A first articulation drive gear 310′ is attached to the articulation drive shaft and is in meshing engagement with a central gear race (not shown) on a second articulation transfer gear (not shown) that is rotatably supported. Thus, rotation of the first articulation drive gear 310′ results in rotation of the second central articulation transfer gear. A “third” articulation shaft gear (not shown) is mounted to a second articulation shaft (not shown) that has a “fourth” articulation worm gear (not shown) thereon. The third articulation shaft gear is in meshing engagement with the second central articulation transfer gear such that rotation of the first articulation drive gear ultimately results in the rotation of the third articulation shaft gear and the second articulation shaft. The fourth articulation worm gear is in meshing engagement with the main articulation gear such that rotation of the fourth articulation worm gear results in rotation of the main articulation drive gear 61A and ultimately application of articulation motions to the articulation link 44. The articulation drive shaft 312′ is rotated by the motor 100′ when the lock 400′ is in an articulation control state.

It is noted that the components and functionality of the motor 100′ and transmission 200′ is substantially the same as that of the motor 100 and transmission 200 of previous examples. Therefore, only germane differences between the example of FIGS. 10-15 and the example of FIGS. 4-5B are discussed further herein. It is further noted that, for the purposes of explanation (and as equivalently discussed with respect to FIG. 4), the ring gears 210′, 230′, 220′ are primarily described as first, second, and third stages as they are arranged sequentially. However, the numbering of the ring gears (and similar components) is non-sequential because, in some examples, and as mentioned elsewhere in the disclosure, the second stage 230′ can be omitted. Thus, the second stage 230′ can also be considered a third, optional stage of the transmission 200.

In the illustrated example, the surgical instrument 10 further includes a firing drive shaft or gear 320′ is connected to the third stage ring gear 220′ at one side thereof, and operably coupled to a third output shaft (not shown) at an opposing side thereof. The third output shaft connects with a third output 61B, such as a firing element, as mentioned above.

A primary articulation drive gear 310′ is attached to the articulation drive shaft 312′ and is in meshing engagement with external teeth of the first stage ring gear 210′. Similarly, a primary closure drive gear 330′ is attached to the closure drive shaft 332′, and which is attached to, e.g., a main closure drive gear 61C) and is in meshing engagement with external teeth of the second stage ring gear 230′.

In this example, the lock 400′ includes a lock shaft 402′ that rotatably supports a first lock 410′ (also referred to herein as a locking pawl), a second lock 430′, and a third lock 420′ that function equivalently as corresponding locks 410, 430, 420 that were previously described. In other words, the locks 410′, 420′, 430′ can be configured as pivotable locking pawls that include teeth that engage the respective outer teeth of gears 210′, 220′, 230′. These locks 410′, 420′, 430′ can be biased to an unlocked position in any appropriate manner, such as a spring or other biasing element.

To pivot the locking pawls 410′, 420′, 430′, the lock 400′ further includes a multi-diameter shaft 404′ (also referred to herein as a locking body) that is repositionable by an indexer 406′. It is noted that, while the term “diameter” is used because the shaft 404′ is depicted with a cylindrical shape, the shaft 404′ is not necessarily cylindrical, and merely requires sections that extend to varying distances from the locks 410′, 420′, 430′ for proper functionality. Therefore, it will be appreciated that the shaft 404′ can also be considered to be a multi-width shaft 404′.

The indexer 406′ is connected to the multi-diameter shaft 404′ via, e.g., a connecting rod 405′ and can include, but is not limited to, a solenoid, a servo motor, or any appropriate linear motion converter. For example, the connecting rod 405′ could be configured as a worm shaft that is rotated by the indexer 406′ to move the multi-diameter shaft 404′ linearly. In examples where the indexer 406′ is embodied as a solenoid, a pair of centering springs 408A′, 408B′ biasing the solenoid to a centered home position.

Distal portions of the pawls 410′, 420′, 430′ engage the outer diameter of the shaft 404′, with the pawls being biased in a direction towards contact with the shaft 404′ to an unlocked position. A first diameter of the shaft 404′ is configured such that its engagement with one of pawls 410′, 420′, 430′ causes rotation of said pawl 410′, 420′, or 430′ to such that proximal portions thereof engage with its respective ring gear 220′ to lock its rotation. A second diameter of the shaft 404′ is configured such that its engagement with one of pawls 410′, 420′, 430′ causes rotation of said pawl 410′, 420′, or 430′ to disengage with its respective ring gear 220′ to unlock its rotation. More specifically, the multi-diameter shaft 404′ can include a first region 404A′ with the first diameter, a second region 404B′ with the first diameter, and a third region 404C′ between the first and second regions 404A′, 404B′ with the second diameter. Moreover, a tapered transition region 404D′ separates the first and third regions 404A′, 404C′ and the second and third regions 404B′, 404C′ to transition between the first and second diameters.

Operation of the lock 400′ can be understood from reference to FIGS. 10-15. Those skilled in the art will appreciate that the exemplary configurations can be modified without departing from the spirit and scope of the present disclosure. For example, the presently described lock 400′ can be adapted for use with a four or more stage geartrain 200′. Moreover, other sequences of varying diameters on the multi-diameter shaft 404′ based on the design requirements of the geartrain 200′. It is further noted that some of these views (e.g., FIGS. 11, 13, and 15), are simplified schematic views that merely depict the structure discussed herein, and not necessarily all of the other structure (e.g., carriers and/or planetary, sun, or other gears) that are otherwise present in the geartrain 200′.

Making reference to FIGS. 10-11, when the indexer 406′ translates (in an axial direction parallel to the longitudinal axis 80′ of the geartrain 200′) the multi-diameter cylinder 404′ and rod 405′ into a first position (e.g., a centered home position), the multi-diameter shaft 404′ can axially align with the geartrain 200′, along the longitudinal axis 80′, such that the first region 404A′ aligns with the first stage ring gear 210′, the second region 404B′ aligns with the third stage ring gear 220′, and the third region 404C′ aligns with second stage ring gear 230′. As seen in the cross-sectional view of FIG. 11, the positioning of the multi-diameter shaft 404′ results in (i) the larger diameter regions 404A′, 404B′ urging both the first lock 410′ (reference number 410′ is shown with dashed lines because it is hidden in the view of FIG. 11) and the second lock 420′, respectively, into a locked position (i.e., the first lock 410′ engages the outer teeth of the first stage ring gear 210′ and the second lock 420′ engages the outer teeth 222′ of the third stage ring gear 220′) and (ii) the smaller diameter region 404C′ permits the third lock 430′ to pivot to an unlocked position (i.e., the third lock 430′ does not engage the outer teeth of the second stage ring gear 230′). This configuration allows the second stage ring gear 230′ to rotate. Thus, upon rotation of motor 100′, the primary closure drive gear 330′ can be driven, while the other output gears 320′, 310′ are prevented from rotation.

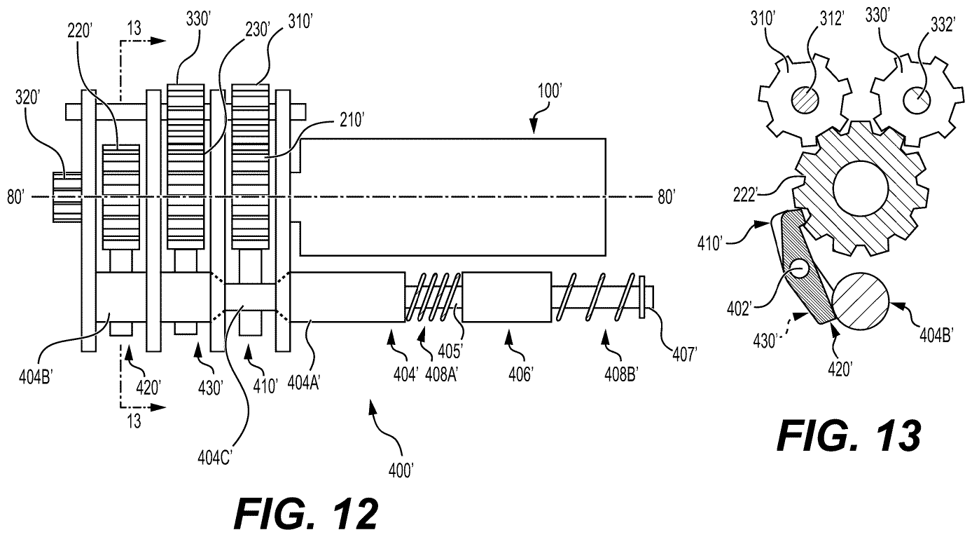

Making reference to FIGS. 12-13, when the indexer 406′ translates (in an axial direction parallel to the longitudinal axis 80′ of the geartrain 200′) the multi-diameter cylinder 404′ and rod 405′ into a second position (e.g., proximally (or to the right) relative to the orientation shown in FIG. 12), the multi-diameter shaft 404′ can axially align with the geartrain 200′, along the longitudinal axis 80′, such that the first region 404A′ is offset (or distanced) from the geartrain 200′, with the first stage ring gear 210′, the second region 404B′ aligns with the third stage ring gear 220′ and the second stage ring gear 430′, and the third region 404C′ aligns with first stage ring gear 210′. As seen in the cross-sectional view of FIG. 13, the positioning of the multi-diameter shaft 404′ results in (i) the larger diameter region 404B′ urging both the second lock 420′ and the third lock 430′, respectively, into a locked position (i.e., the second lock 420′ engages the outer teeth 222′ of the third stage ring gear 220′ and the third lock 430′ (reference number 430′ is shown with dashed lines because it is hidden in the view of FIG. 13) engages the outer teeth of the second stage ring gear 230′) and (ii) the smaller diameter region 404C′ permits the first lock 410′ to pivot to an unlocked position (i.e., the first lock 410′ does not engage the outer teeth of the first stage ring gear 210′). This configuration allows the first stage ring gear 210′ to rotate. Thus, upon rotation of motor 100′, the primary closure drive gear 330′ can be driven, while the other output gears 320′, 310′ are prevented from rotation.

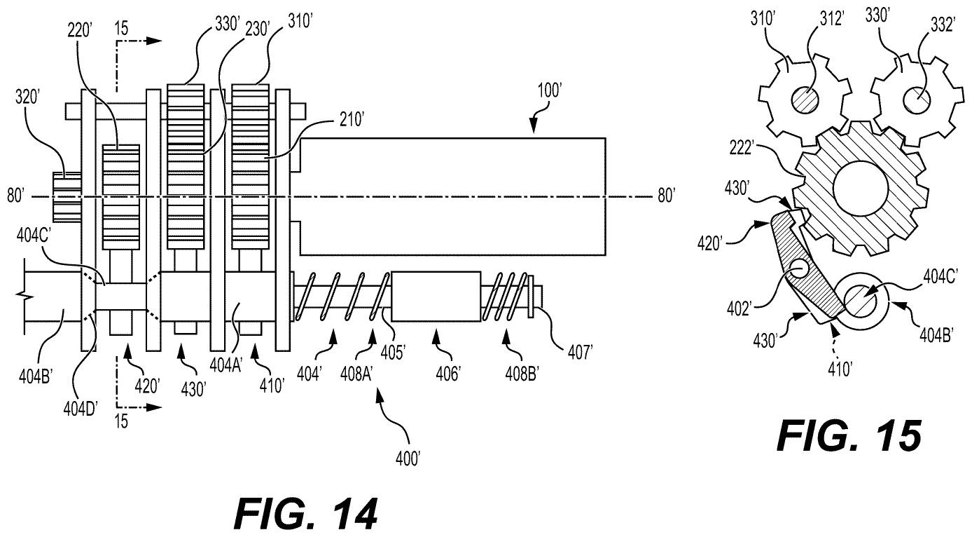

Making reference to FIGS. 14-15, when the indexer 406′ translates (in an axial direction parallel to the longitudinal axis 80′ of the geartrain 200′) the multi-diameter cylinder 404′ and rod 405′ into a third position (e.g., distally (or to the left) relative to the orientation shown in FIG. 12), the multi-diameter shaft 404′ can axially align with the geartrain 200′, along the longitudinal axis 80′, such that the first region 404A′ aligns with the first stage ring gear 210′ and the second stage ring gear 230′, the second region 404B′ is offset (or distanced) from the geartrain 200′, and the third region 404C′ aligns with third stage ring gear 220′. As seen in the cross-sectional view of FIG. 15, the positioning of the multi-diameter shaft 404′ results in (i) the larger diameter region 404A′ urging both the first lock 410′ (reference number 410′ is shown with dashed lines because it is hidden in the view of FIG. 15) and the third lock 430′, respectively, into a locked position (i.e., the first lock 410′ engages the outer teeth of the first stage ring gear 210′ and the third lock 430′ engages the outer teeth of the second stage ring gear 230′) and (ii) the smaller diameter region 404C′ permits the second lock 420′ to pivot to an unlocked position (i.e., the second lock 420′ does not engage the outer teeth 222′ of the third stage ring gear 220′). This configuration allows the third stage ring gear 220′ to rotate. Thus, upon rotation of motor 100′, the firing drive gear 320′ can be driven, while the other output gears 310′, 330′ are prevented from rotation.

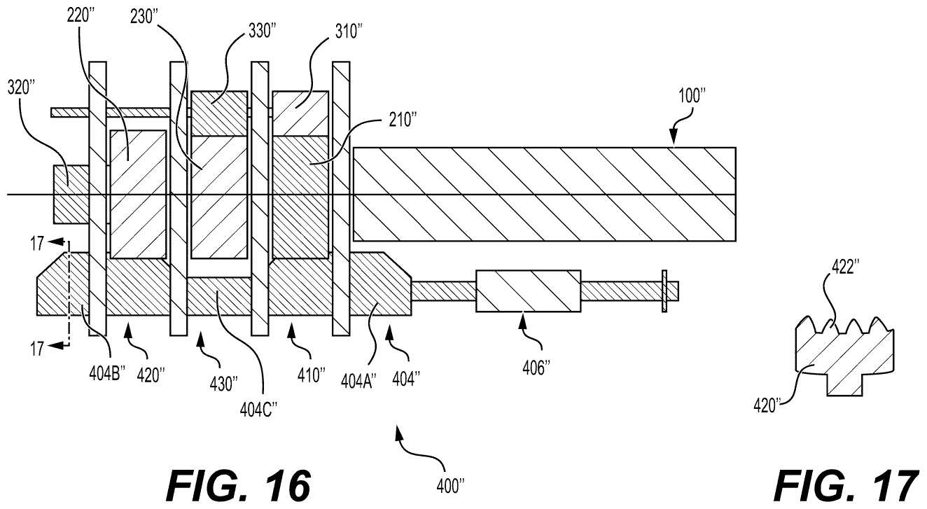

FIGS. 16-17 depict yet another configuration of transmission and lock assembly 200″, 400″ in accordance with the present disclosure. This example can be configured and function identically as the example of FIGS. 10-15, except where noted below, with equivalent components being marked with two apostrophes in place of a single apostrophe. Therefore, only the differences between the two examples are discussed herein. In this example, the respective first, second, and third stage ring gears 310″, 330″, 320″ are selectively lockable and unlockable by a lock 400″ that includes a translating locking block 404″ (also referred to herein as a locking body). Like the previously described multi-diameter barrel 404′, the locking block 404″ is translatable by an indexer 404″. Rather than providing separate pawls 410′, 420′, 430′ that are actuated by a shaft 404′, the locking block 404″ in this example directly engages the ring gears 310″, 330″, 320″. The locking block 404″ can include (i) a pair of regions 404A″, 404B″ that include teeth 422″ (or other structure for preventing gear rotation) that each can selectively mesh with at least one of the ring gears 310″, 330″, 320″ to lock rotation thereof and (ii) another region 404C″ in between the aforementioned pair of regions 404A″, 404B″ that does not mesh with any of the ring gears 310″, 330″, 320″. In other words, a single structure (the locking block 404″) can be used to achieve the same functionality as that of the pawls 410′, 420′, 430′ and shaft 404′ described in the previous example.

The surgical instrument systems described herein have been described in connection with the deployment and deformation of staples; however, the embodiments described herein are not so limited. Various embodiments are envisioned which deploy fasteners other than staples, such as clamps or tacks, for example. Moreover, various embodiments are envisioned which utilize any suitable means for sealing tissue. For instance, an end effector in accordance with various embodiments can comprise electrodes configured to heat and seal the tissue. Also, for instance, an end effector in accordance with certain embodiments can apply vibrational energy to seal the tissue.

Although various devices have been described herein in connection with certain embodiments, modifications and variations to those embodiments may be implemented. Particular features, structures, or characteristics may be combined in any suitable manner in one or more embodiments. Thus, the particular features, structures, or characteristics illustrated or described in connection with one embodiment may be combined in whole or in part, with the features, structures or characteristics of one ore more other embodiments without limitation. Also, where materials are disclosed for certain components, other materials may be used. Furthermore, according to various embodiments, a single component may be replaced by multiple components, and multiple components may be replaced by a single component, to perform a given function or functions. The foregoing description and following claims are intended to cover all such modification and variations.

The disclosed technology described herein can be further understood according to the following clauses:
Clause 1. A transmission for a motor-powered device, the transmission comprising: a motor input (62); a first output (61A) drivable by the motor input (62); a second output (61B) drivable by the motor input (62); a planetary gearbox (63) operably connecting the motor input with the first output and the second output and comprising: a first stage ring gear (63A) engaged with the first output; a second stage ring gear (63B) engaged with the second output; and a lock (67) selectively engageable with at least one of the first stage ring gear (63A) or the second stage ring gear (63B), the lock having (i) an unlocked position allowing the at least one of the first stage ring gear (63A) or the second stage ring gear (63B) to power at least one of the first output (61A) or the second output (61B) and (ii) a locked position that stops rotation of the at least one of the first stage ring gear (63A) or the second stage ring gear (63B) to lock at least one of the first output (61A) or the second output (61B).
Clause 2. The transmission of clause 1, wherein the second output (61B) comprises a firing control and the first output (61A) comprises an articulation control.
Clause 3. The transmission of any one of clauses 1-2, wherein the motor-powered device comprises a surgical instrument (10).
Clause 4. The transmission of any one of clauses 1-3, wherein the lock (67) directly engages the at least one of the first stage ring gear (63A) or the second stage ring gear (63B).
Clause 5. The transmission of any one of clauses 1-4, further comprising: a first drive gear (310) meshed with the first stage ring gear (210); and a first output shaft (312) connected to (i) the first output (61A) and (ii) the first drive gear (310) such that rotation of the first drive gear (310) causes rotation of the first output shaft (312).
Clause 6. The transmission of any one of clauses 1-5, further comprising: a second output shaft (322) connected to (i) the second output (61B) and (ii) the second stage ring gear (220) such that rotation of the second stage ring gear (220) causes rotation of the second output shaft (322).
Clause 7. The transmission of clause 6, wherein the second output shaft (322) extends along a first axis (80) and the first output shaft (312) extends along a second axis (82) that is parallel to the first axis (80).
Clause 8. The transmission of any one of clauses 1-7, wherein the first stage ring gear (210) comprises a plurality of inner teeth (212) and a plurality of outer teeth (214) opposed to one another in a radial direction of the first stage ring gear (210).
Clause 9. The transmission of any one of clauses 1-8, wherein the lock (400) comprises: a first lock (410) selectively engageable with the first stage ring gear (210), the first lock having (i) an unlocked position allowing the first stage ring gear (210) to power the first output (61A) and (ii) a locked position that stops rotation of the first stage ring gear (210) to lock the first output (61A); and a second lock (420) selectively engageable with the second stage ring gear (222) and independently controllable from the first lock (410), the second lock (420) having (i) an unlocked position allowing the second stage ring gear (222) to power the second output (61B) and (ii) a locked position that stops rotation of the second stage ring gear (222) to lock the second output (61B).
Clause 10. The transmission of any one of clauses 1-9, wherein the lock (400) comprises a locking pawl (410) pivotable between the unlocked position and the locked position.
Clause 11. The transmission of any one of clauses 1-9, further comprising: at least one magnet (211A) associated with the at least one of the first stage ring gear (210A) or the second stage ring gear (220), wherein the lock (410A) comprises at least one electromagnetic resistance coil (412A) configured to induce electromagnetic forces that at least one of attract or repel the at least one magnet (211A).
Clause 12. The transmission of clause 11, wherein the at least one electromagnetic resistance coil (412B, 412E) comprises a plurality of independently controllable electromagnetic resistance coils (412B, 412E).
Clause 13. The transmission of any one of clauses 11-12, wherein the at least one magnet (211A) is embedded in the at least one of the first stage ring gear (210A) or the second stage ring gear (220).
Clause 14. The transmission of any one of clauses 11-13, further comprising a first drive gear (310C) meshed with the first stage ring gear (210C), wherein the at least one magnet (311C) is embedded in the first drive gear (310C).
Clause 15. The transmission of any one of clauses 11-14, further comprising a spur gear (311D) meshed with the first stage ring gear (310D), wherein the at least one magnet (311D1) is embedded in the spur gear (311D).
Clause 16. The transmission of any one of clauses 1-15, further comprising: a third output (61C) drivable by the motor input (62); a third stage ring gear (230) engaged with the third output (61C), wherein the lock (400) is selectively engageable with the third stage ring gear (230), the lock having (i) an unlocked position allowing the third stage ring gear (230) to power the third output (61C) and (ii) a locked position that stops rotation of the third stage ring gear (230) to lock the third output (61C).
Clause 17. The transmission of clause 16, wherein the third stage ring gear (230) is interposed between the first stage ring gear (210) and the second stage ring gear (220).
Clause 18. The transmission of any one of clauses 15-17, wherein the third stage ring gear (230) comprises a plurality of inner teeth (232) and a plurality of outer teeth (234) opposed to one another in a radial direction of the first stage ring gear (210).
Clause 19. The transmission of clause 18, further comprising a third drive gear (330) meshed with the plurality of outer teeth (234) of the third stage ring gear (230).
Clause 20. The transmission of any one of clauses 15-19, wherein the third output (61C) comprises a closure control.
Clause 21. The transmission of any one of clauses 1-7, wherein a single lock (910) is selectively engageable with the first stage ring gear (710) and the second stage ring gear (720) such that movement to the locked position of the first stage ring gear (710) unlocks the second stage ring gear (720), and movement to the unlocked position of the first stage ring gear (710) locks the second stage ring gear (720).
Clause 22. The transmission of any one of clauses 1-7 and 21, wherein the lock (910) is translatable between the locked position and unlocked position.
Clause 23. The transmission of clause 22, further comprising a control (920) including an engagement surface (922), wherein the lock (910) comprises a cam surface (914) configured to interact with the engagement surface (922) to translate the lock (910).
Clause 24. The transmission of clause 23, further comprising a spring (930) engaged with the lock (910), wherein the spring (930) is configured to bias the lock (910) in a first direction, and the engagement surface (922) is configured to translate the lock (910) in a second, opposite direction.
Clause 25. The transmission of any one of clauses 1-7 and 21-24, wherein the first stage ring gear (710) comprises a first set of outer teeth (714) and a second set of outer teeth (716) oriented perpendicularly relative to one another.
Clause 26. The transmission of clause 25, further comprising: a first drive gear (810) meshed with the first stage ring gear; and a first output shaft (812) extending along a second axis (782), wherein the second set of outer teeth extend from a lateral side (717) of the first stage ring gear and are parallel with the second axis.
Clause 27. The transmission of any one of clauses 25-26, wherein the second stage ring gear (720) comprises teeth (722) that face towards the second set of outer teeth of the first stage ring gear.
Clause 28. The transmission of clause 27, wherein the lock comprises a first set of teeth (912) configured to mesh with the second set of outer teeth (716) of the first stage ring gear (710) to lock the first stage ring gear (710) and a second set of teeth (913) configured to mesh with the teeth (722) of the second stage ring gear (720) to lock the second stage ring gear (720).
Clause 29. The transmission of clause 8, wherein the lock (400′, 400″) comprises a locking body (404′, 404″) that is translatable, along an axis parallel to a longitudinal axis (80′) of the first stage ring gear (210′, 210″) and the second stage ring gear (220′, 220″) between (i) a first position where the first stage ring gear (210′, 210″) is allowed to rotate to power the first output (61A) and where the second stage ring gear (220′, 220″) is prevented from rotation to lock the second output (61B) and (ii) a second position where the first stage ring gear (210′, 210″) is prevented from rotation to lock the first output (61A) and where the second stage ring gear (220′, 220″) is allowed to rotate to power the second output (61B).
Clause 30. The transmission of claim 29, further comprising a further comprising: a third output (61C) drivable by the motor input (62); a third stage ring gear (230′, 230″) engaged with the third output (61C), wherein the locking body is translatable between the first position, the second position, and a third position where the first stage ring gear (210′, 210″) is prevented from rotation to lock the first output (61A), the second stage ring gear (220′, 220″) is prevented from rotation to lock the second output (61B), and the third stage ring gear (230′, 230″) is allowed to rotate to power the third output (61C).
Clause 31. The transmission of clause 30, wherein the lock (400′) comprises: a first locking pawl (410′) pivotable between the unlocked position and the locked position to selectively engage the outer teeth to unlock or lock rotation of the first stage ring gear (210′); a second locking pawl (420′) pivotable between the unlocked position and the locked position to selectively engage outer teeth of the second stage ring gear (220′) unlock or lock rotation of the second stage ring gear (220′); and a third locking pawl (430′) pivotable between the unlocked position and the locked position to selectively engage outer teeth of the third stage ring gear (230′) to unlock or lock rotation of the third stage ring gear (230′), wherein the locking body (404′) comprises a multi-diameter shaft (404′) comprising a plurality of regions (404A′, 404B′, 404C′) configured to engage the first locking pawl (410′), the second locking pawl (420′), and the third locking pawl (430′) to respectively move the first locking pawl (410′), the second locking pawl (420′), and the third locking pawl (430′) between the unlocked position and the locked position.
Clause 32. The transmission of clause 31, wherein the plurality of regions comprises a first region (404A′), a second region (404B′), and a third region (404C′) disposed between the first region (404A′) and the second region (404B′), the first region and the second region comprising a first diameter and the third region comprising a second diameter, less than the first diameter.
Clause 33. The transmission of clause 30, wherein the locking body (404′) comprises a locking block (404″) comprising first region (404A″), a second region (404B″), and a third region (404C″) disposed between the first region (404A″) and the second region (404B″), the first region (404A″) and the second region (404B″) each being configured to simultaneously directly engage one or more of the first stage ring gear (210″), the second stage ring gear (220″), or the third stage ring gear (230″′) to prevent rotation of the one or more of the first stage ring gear (210″), the second stage ring gear (220″), or the third stage ring gear (230″). Clause 34. The transmission of clause 33, wherein: in the first position, the second region (404C″) is aligned the first stage ring gear (210″) and is spaced therefrom to allow rotation of the first stage ring gear (210″), in the second position, the second region (404C″) is aligned the second stage ring gear (220″) and is spaced therefrom to allow rotation of the second stage ring gear (220″), and in the third position, the second region (404C″) is aligned the third stage ring gear (230″) and is spaced therefrom to allow rotation of the third stage ring gear (230″),
Clause 35. The transmission of any one of clause 33-34, wherein the first region (404A″) and the second region (404B″) comprise gear teeth.
Clause 36. A surgical instrument comprising the transmission (200, 200′, 200″, 700) of any one of clauses 1-35.
Clause 37. The surgical instrument of clause 36, wherein the surgical instrument (10) comprises an endocutter.
Clause 38. A method (1000) of operating a motor-powered device comprising: controlling (1002) a lock such that the lock is engaged with a first stage ring gear and disengaged with a second stage ring gear; powering (1004) a first output engaged with the first stage ring gear; locking (1006) a second output engaged with the second stage ring gear; controlling (1008) the lock such that the lock is engaged with the second stage ring gear and disengaged with the first stage ring gear; powering (1010) the second output; and locking (1012) the first output.
Clause 39. The method of clause 38, further comprising: controlling the lock such that the lock is disengaged with the second stage ring gear and disengaged with the first stage ring gear; and powering the first output and the second output simultaneously.

While this technology has been described as having exemplary designs, the present disclosure may be further modified within the spirit and scope of the disclosure. This application is therefore intended to cover any variations, uses, or adaptations of the presently disclosed technology using its general principles.

What is claimed is:

1. A transmission for a motor-powered device, the transmission comprising:
a motor input;
a first output drivable by the motor input;
a second output drivable by the motor input; and
a planetary gearbox operably connecting the motor input with the first output and the second output and comprising:
a first stage ring gear engaged with the first output;
a second stage ring gear engaged with the second output; and
a lock selectively engageable with at least one of the first stage ring gear or the second stage ring gear, the lock having (i) an unlocked position allowing the at least one of the first stage ring gear or the second stage ring gear to power at least one of the first output or the second output and (ii) a locked position that stops rotation of the at least one of the first stage ring gear or the second stage ring gear to lock at least one of the first output or the second output.

2. The transmission of claim 1, wherein the second output comprises a firing control and the first output comprises an articulation control.

3. The transmission of claim 1, wherein the motor-powered device comprises a surgical instrument.

4. The transmission of claim 1, wherein the lock directly engages the at least one of the first stage ring gear or the second stage ring gear.

5. The transmission of claim 1, further comprising:
a first drive gear meshed with the first stage ring gear; and
a first output shaft connected to (i) the first output and (ii) the first drive gear such that rotation of the first drive gear causes rotation of the first output shaft.

6. The transmission of claim 5, further comprising:
a second output shaft connected to (i) the second output and (ii) the second stage ring gear such that rotation of the second stage ring gear causes rotation of the second output shaft.

7. The transmission of claim 6, wherein the second output shaft extends along a first axis and the first output shaft extends along a second axis that is parallel to the first axis.

8. The transmission of claim 1, wherein the first stage ring gear comprises a plurality of inner teeth and a plurality of outer teeth opposed to one another in a radial direction of the first stage ring gear.

9. The transmission of claim 8, wherein the lock comprises a locking body that is translatable, along an axis parallel to a longitudinal axis of the first stage ring gear and the second stage ring gear between (i) a first position where the first stage ring gear is allowed to rotate to power the first output and where the second stage ring gear is prevented from rotation to lock the second output and (ii) a second position where the first stage ring gear is prevented from rotation to lock the first output and where the second stage ring gear is allowed to rotate to power the second output.

10. The transmission of claim 1, wherein the lock comprises:
a first lock selectively engageable with the first stage ring gear, the first lock having (i) an unlocked position allowing the first stage ring gear to power the first output and (ii) a locked position that stops rotation of the first stage ring gear to lock the first output; and
a second lock selectively engageable with the second stage ring gear and independently controllable from the first lock, the second lock having (i) an unlocked position allowing the second stage ring gear to power the second output and (ii) a locked position that stops rotation of the second stage ring gear to lock the second output.

11. The transmission of claim 1, wherein the lock comprises a locking pawl pivotable between the unlocked position and the locked position.

12. The transmission of claim 1, further comprising:
at least one magnet associated with the at least one of the first stage ring gear or the second stage ring gear, wherein
the lock comprises at least one electromagnetic resistance coil configured to induce electromagnetic forces that at least one of attract or repel the at least one magnet.

13. The transmission of claim 12, wherein the at least one electromagnetic resistance coil comprises a plurality of independently controllable electromagnetic resistance coils.

14. The transmission of claim 12, wherein the at least one magnet is embedded in the at least one of the first stage ring gear or the second stage ring gear.

15. The transmission of claim 1, wherein a single lock is selectively engageable with the first stage ring gear and the second stage ring gear such that movement to the locked position of the first stage ring gear unlocks the second stage ring gear, and movement to the unlocked position of the first stage ring gear locks the second stage ring gear.

16. The transmission of claim 1, wherein the lock is translatable between the locked position and unlocked position.

17. The transmission of claim 16, further comprising a control including an engagement surface, wherein
the lock comprises a cam surface configured to interact with the engagement surface to translate the lock.

18. The transmission of claim 17, further comprising a spring engaged with the lock, wherein
the spring is configured to bias the lock in a first direction, and
the engagement surface is configured to translate the lock in a second, opposite direction.

19. A surgical instrument comprising a transmission comprising:
a motor input;
a first output of the surgical instrument that is drivable by the motor input;
a second output of the surgical instrument that is drivable by the motor input; and
a planetary gearbox operably connecting the motor input with the first output and the second output and comprising:
a first stage ring gear engaged with the first output;
a second stage ring gear engaged with the second output; and
a lock selectively engageable with at least one of the first stage ring gear or the second stage ring gear, the lock having (i) an unlocked position allowing the at least one of the first stage ring gear or the second stage ring gear to power at least one of the first output or the second output and (ii) a locked position that stops rotation of the at least one of the first stage ring gear or the second stage ring gear to lock at least one of the first output or the second output.

20. A method of operating a motor-powered device comprising:
controlling a lock such that the lock is engaged with a first stage ring gear and disengaged with a second stage ring gear;
powering a first output engaged with the first stage ring gear;
locking a second output engaged with the second stage ring gear;
controlling the lock such that the lock is engaged with the second stage ring gear and disengaged with the first stage ring gear;
powering the second output; and
locking the first output.
